(19) United States
(12) Patent Application Publication
(10) Pub. No.: US 2021/0338846 A1
(43) Pub. Date: Nov. 4, 2021

(54) COMPETITIVE PROSTATE-SPECIFIC MEMBRANE ANTIGEN (PSMA) BINDING AGENTS FOR REDUCTION OF NON-TARGET ORGAN UPTAKE OF RADIOLABELED PSMA INHIBITORS FOR PSMA POSITIVE TUMOR IMAGING AND RADIOPHARMACEUTICAL THERAPY

(71) Applicant: The Johns Hopkins University, Baltimore, MD (US)

(72) Inventors: Martin G. Pomper, Baltimore, MD (US); Ronnie C. Mease, Fairfax, VA (US); Sangeeta Ray, Ellicott City, MD (US); IL Minn, Ellicott City, MD (US)

(21) Appl. No.: 17/264,222

(22) PCT Filed: Jul. 30, 2019
(86) PCT No.: PCT/US2019/044074

§ 371 Date: Jan. 28, 2021

Related U.S. Application Data
(60) Provisional application No. 62/711,735, filed on Jul. 30, 2018.

(51) Int. Cl. A61K 51/04; A61K 31/198; A61K 31/27; A61K 31/455; A61P 35/00

(52) U.S. Cl. CPC A61K 51/0402; A61K 31/198; A61P 35/00; A61K 31/455; A61K 31/27

(57) ABSTRACT

Methods for co-injection of a non-radioactive PSMA inhibitor, referred to herein as a competing inhibitor (CI), with a radiolabeled PSMA inhibitor are disclosed. This combination reduces the uptake of the radiotracer in non-target organs, including the kidneys and lacrimal glands, with only a modest reduction in tumor uptake.

### FEDERALLY SPONSORED RESEARCH OR DEVELOPMENT

This invention was made with government support under CA134675 awarded by the National Institutes of Health. The government has certain rights in the invention.

### BACKGROUND

Prostate-specific membrane antigen (PSMA), also known as glutamate carboxypeptidase II (GCPII), N-acetyl-L-aspartyl-L-glutamate peptidase I (NAALADase I), or NAAG peptidase, is a marker for androgen-independent disease that also is expressed on solid (non-prostate) tumor neovasculature. The synthesis of a variety of high affinity, radiolabeled (e.g., radiohalogen- or radiometal-labeled) urea-based PSMA inhibitors that selectively visualize prostate cancer (PCa) tumors in experimental models has been reported previously. Two of these compounds, [¹⁸F]DCFBC and [¹⁸F]DCFPyL, have been translated to clinical studies where they have been shown to successfully detect primary and metastatic PCa in patients using positron emission tomography (PET)/computed tomography (CT).

[¹⁸F]DCFPyL also has been able to detect metastatic clear cell renal cell carcinoma (ccRCC) due to the high vascularity of these lesions. PSMA-targeting agents labeled with radiotherapeutic nuclides also have demonstrated effectiveness in treating metastatic PCa. Because PSMA also is expressed in certain healthy organs, such as the kidney, lacrimal glands, and salivary glands, although in lower concentrations than PCa, PSMA-based agents also localize in these non-target tissues. While this non-target tissue uptake does not affect PCa imaging because PCa rarely metastasizes to the kidney, a method of reducing renal uptake and retention would potentially allow detection of primary ccRCC. In addition, decreasing the non-target tissue uptake would reduce the undesirable side effects of PSMA-targeted radiopharmaceutical therapy (RPT), including renal toxicity and dry mouth, especially in RPT using alpha emitters. Reducing the initial renal uptake is especially important when using short-lived alpha emitting radionuclides, such as At-211 (T_{1/2}=7.21H).

### SUMMARY

In some aspects, the presently disclosed subject matter provides a method for imaging a prostate-specific membrane antigen (PSMA)-positive tumor or cell or treating a disease, disorder, or condition associated with PSMA, the method comprising administering to the subject a radiolabeled PSMA inhibitor in combination with a non-radiolabeled PSMA competing inhibitor, each in an amount suitable for imaging a PSMA-positive tumor or treating a disease, disorder, or condition associated with PSMA.

In particular aspects, the prostate-specific membrane antigen (PSMA)-positive tumor or cell is primary clear cell renal carcinoma.

In other aspects, the method further comprises taking an image. In certain aspects, the taking of an image is selected from the group consisting of positron emission tomography (PET) and single-photon emission computed tomography (SPECT).

In other aspects, the presently disclosed subject matter provides a pharmaceutical composition comprising a radiolabeled PSMA inhibitor in combination with a non-radiolabeled PSMA competing inhibitor.

In yet other aspects, the presently disclosed subject matter provides a kit comprising a radiolabeled PSMA inhibitor in combination with a non-radiolabeled PSMA competing inhibitor.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

The patent or application file contains at least one drawing executed in color. Copies of this patent or patent application publication with color drawings will be provided by the Office upon request and payment of the necessary fee.

Having thus described the presently disclosed subject matter in general terms, reference will now be made to the accompanying Figures, which are not necessarily drawn to scale, and wherein:

FIG. 1A, FIG. 1B, FIG. 1C, and FIG. 1D show that treatment with [²¹¹At]DCIBzL caused severe renal damage. Renal histopathology from nontreated mouse (FIG. 1A and FIG. 1B) and mouse treated with 1.5 MBq of [²¹¹At]DCIBzL (FIG. 1C and FIG. 1D). Treated kidney showed subcortical atrophy and degenerative loss of proximal tubules (arrows) consistent with late nephropathy due to α-particle irradiation. FIG. 1A, FIG. 1C: 2×; FIG. 1B, FIG. 1D: 10×. (from Kiess et al., 2016; prior art); and

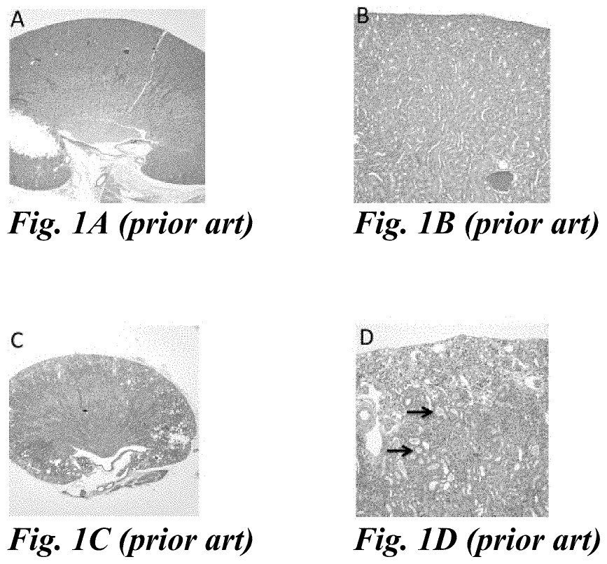

FIG. 2 demonstrates that co-injection of cold YC-I-27 (DCIBzL) selectively blocks the kidney uptake of [¹²⁵I]VK-02-90-Lu. Mice carrying PSMA+ subcutaneous tumor were co-injected with 37 kBq of [¹²⁵I]VK-02-90-Lu with selected cold blockers. The uptake levels of [¹²⁵I]VK-02-90-Lu from indicated organs were measured at the indicated time points. YC-1-27 (DCIBzL) selectively and significantly blocks the uptake of [¹²⁵I]VK-02-90-Lu from kidney at early (1 hr) time point.

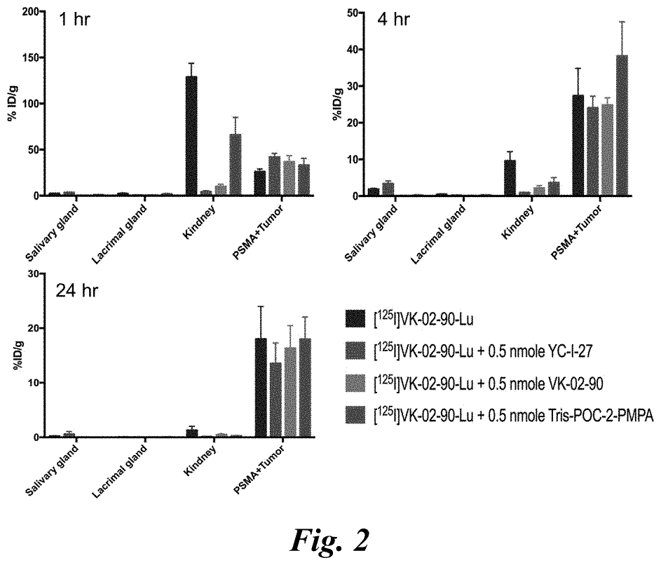

### DETAILED DESCRIPTION

The presently disclosed subject matter now will be described more fully hereinafter with reference to the accompanying Figures, in which some, but not all embodiments of the presently disclosed subject matter are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

### I. Competitive PSMA Binding Agents for the Reduction of Non-Target Organ Uptake of Radiolabeled PSMA Inhibitors for PSMA Positive Tumor Imaging and Radiopharmaceutical Therapy

The presently disclosed subject matter provides, in part, methods for co-injection of a non-radioactive PSMA inhibitor, referred to herein as a competing inhibitor (CI), with a radiolabeled PSMA inhibitor. This combination reduces the uptake of the radiotracer, i.e., the radiolabeled PSMA inhibitor, in non-target organs, including the kidneys, lacrimal glands, and salivary glands, with only a modest reduction in tumor uptake. This characteristic is an important aspect in extending PSMA-based imaging to primary clear cell renal carcinoma and reducing the undesirable side effects of PSMA-targeted radiopharmaceutical therapy, including, but not limited to, renal toxicity and dry mouth. Further, the characteristics of an effective CI is that it has a high initial renal uptake, which can be determined by the biodistribution in mice of its radiolabeled version.

A. Methods for Imaging a Prostate-Specific Membrane Antigen (PSMA) Positive Tumor or Treating a Disease, Disorder, or Condition Associated with PSMA

Accordingly, in some embodiments, the presently disclosed subject matter provides a method for imaging a prostate-specific membrane antigen (PSMA)-positive tumor or treating a disease, disorder, or condition associated with PSMA, the method comprising administering to the subject a radiolabeled PSMA inhibitor in combination with a non-radiolabeled PSMA competing inhibitor, each in an amount suitable for imaging a PSMA-positive tumor or treating a disease, disorder, or condition associated with PSMA.

In some embodiments, the competing inhibitor is YC-I-27, also referred to herein as DCIBzL:

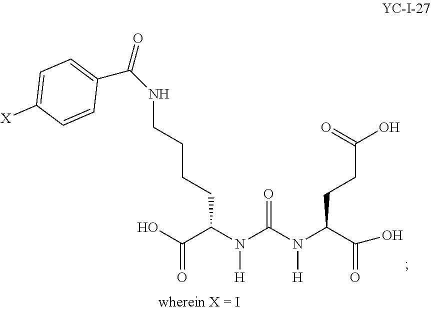

YC-I-27 was first reported by in 2008 (Y. Chen et al. J. Med. Chem. 51:7933-7933, 2008) as an imaging agent and subsequently disclosed in international PCT patent application publication no. WO2008058192 A2, to Babich et al., which is incorporated herein by reference in its entirety.

In other embodiments, the competing inhibitor is a derivative or analogue of YC-I-27 of formula (I) as disclosed in international PCT patent application publication no. WO2008058192 A2, to Babich et al.:

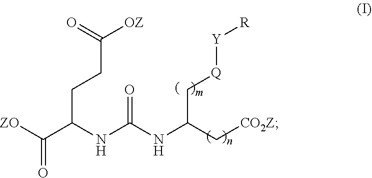

wherein:

R is a C₆-C₁₂ substituted or unsubstituted aryl, a C₆-C₁₂ substituted or unsubstituted heteroaryl, a C₁-C₆ substituted or unsubstituted alkyl, or —NR′R′:

Q is C(O), O, NR′, S, S(O)₂, C(O)₂, or (CH₂)ₚ;

Y is C(O), O, NR′, S, S(O)₂, C(O)₂, or (CH₂)ₚ;

Z is H or C₁-C₄ alkyl;

m is 0, 1, 2, 3, 4 or 5;

n is 0, 1, 2, 3, 4, 5 or 6;

p is 0, 1, 2, 3, 4, 5 or 6;

R′ is H, C(O), S(O)₂, C(O)₂, a C₆-C₁₂ substituted or unsubstituted aryl, a C₆-C₁₂ substituted or unsubstituted heteroaryl or a C₁-C₆ substituted or unsubstituted alkyl, when substituted, aryl, heteroaryl and alkyl are substituted with halogen, C₆-C₁₂ heteroaryl, —NR′R′ or COOZ;

further wherein: (i) at least one of R or R′ is a C₆-C₁₂ aryl or C₆-C₁₂ heteroaryl substituted with a halogen or (ii) at least one of R or R′ is a C₆-C₁₂ heteroaryl;

or a pharmaceutically acceptable salt thereof.

In particular embodiments of the compound of formula (I) from Babich et al., n is 0 or 1; m is 0, 1, 2, 3 or 4; Q is NR′; Y is C(O) or CH₂; and R is a C₆-C₁₂ substituted or substituted aryl. In yet more particular embodiments, R is a phenyl moiety substituted with a halogen.

In other embodiments, the competing inhibitor is a compound similar to YC-I-27 having a 4-substituted, 3-substituted-, or 2-fluoro-4-substituted benzoyl group, including compounds of formula (XX-A):

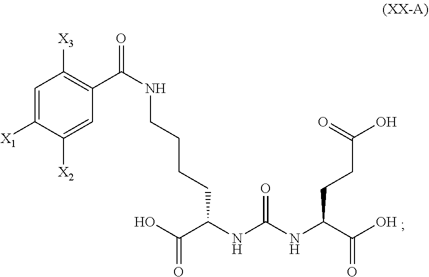

wherein: X₁ and X₂ are each independently selected from the group selected from Br, Cl, I, F, H, —OCH₃, —OCH₂CH₃, —OCH(CH₃)₂, and —OC(CH₃)₃. In particular embodiments, compounds 1-23 are as follows:

Compounds 1-23

| Compound | X₁ | X₂ | X₃ |
|---|---|---|---|
| 1 | Br | H | H |
| 2 | Cl | H | H |
| 3 | F | H | H |
| 4 | H | H | H |
| 5 | —OCH₃ | H | H |
| 6 | —OCH₂CH₃ | H | H |
| 7 | —OCH(CH₃)₂ | H | H |
| 8 | —OC(CH₃)₃ | H | H |
| 9 | H | I | H |
| 10 | H | Br | H |
| 11 | H | Cl | H |
| 12 | H | F | H |
| 13 | H | —OCH₃ | H |
| 14 | H | —OCH₂CH₃ | H |
| 15 | H | —OCH(CH₃)₂ | H |
| 16 | H | —OC(CH₃)₃ | H |
| 17 | Br | H | F |
| 18 | I | H | F |
| 19 | Cl | H | F |
| 20 | —OCH₃ | H | F |
| 21 | —OCH₂CH₃ | H | F |
| 22 | —OCH(CH₃)₂ | H | F |
| 23 | —OC(CH₃)₃ | H | F |

In other embodiments, the competing inhibitor is a reverse carbamate of formula (XX-B):

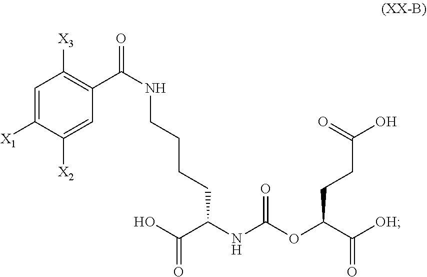

wherein: X₃ is F, X₂ is H, and X₁ is selected from the group consisting of Br, Cl, I, —OCH₃, —OCH₂CH₃, —OCH(CH₃), and —OC(CH₃)₃. Representative reverse carbamates suitable for use with the presently disclosed subject matter include those compounds disclosed in international PCT patent application publication no. WO2016065145 A2, to Pomper et al., which is incorporated herein by reference in its entirety).

In particular embodiments, compounds 24-30 are as follows:

Compounds 24-30

| Compound | X₁ | X₂ | X₃ |
|---|---|---|---|
| 24 | I | H | F |
| 25 | Br | H | F |
| 26 | Cl | H | F |
| 27 | —OCH₃ | H | F |
| 28 | —OCH₂CH₃ | H | F |
| 29 | —OCH(CH₃)₂ | H | F |
| 30 | —OC(CH₃)₃ | H | F |

In some embodiments, the competing inhibitor is a 4- or 5-halo-nicotinamide-lysine-glutamine urea. Representative halo-nicotimamides of formula (XX-C) include, but are not limited to, compound 31 and compound 38 (also referred to herein as DCFPyL), which were first reported by Chen et al. J. Med. Chem. 51: 7933-7943, 2008; Clinical Cancer research 17: 7645-7653, 2011), as well as analogs 32-37:

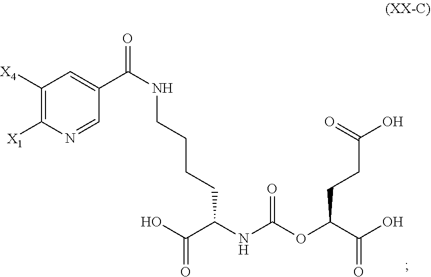

Wherein X₁ and X₄ are each independently selected from the group consisting of H, I, Br, Cl, and F.

| Compound | X₁ | X₄ |
|---|---|---|
| 31 | H | I |
| 32 | H | Br |
| 33 | H | Cl |
| 34 | H | F |
| 35 | I | H |
| 36 | Br | H |
| 37 | Cl | H |
| 38 | F | H |

In some embodiments, the competing inhibitor is HS-549 or an HS-549 analog. Compound 39 (HS-549) and its analogs compounds 40-58 were first reported in international PCT patent application publication no. WO 2017070482 A2, to Pomper et al.

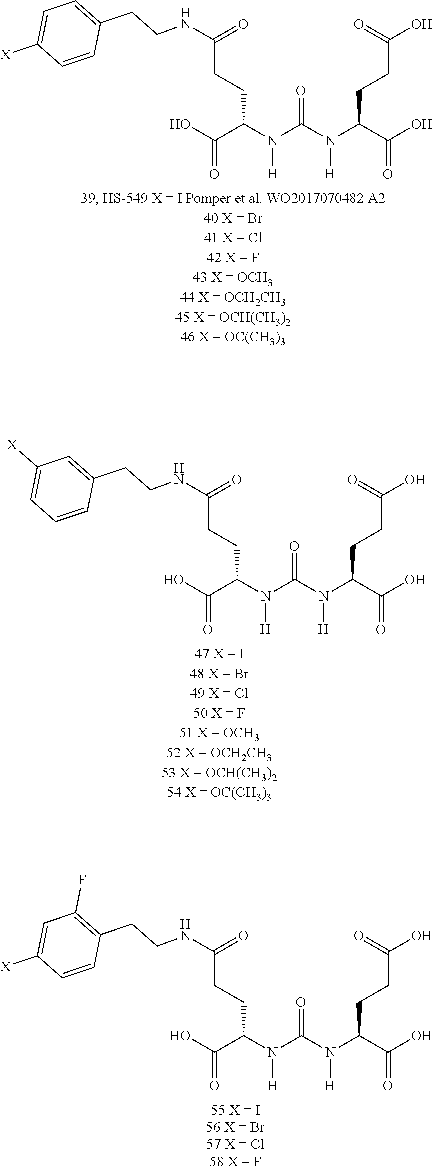

In other embodiments, the competing inhibitor is selected from the group consisting of PSMA-620, PSMA-904, and analogs thereof. PSMA-620 and PSMA-904 are disclosed in international PCT patent application publication no. WO 2017070482 A2, to Pomper et al., which is incorporated herein by reference in its entirety. PSMA-620 and PSMA-904 each demonstrated high uptake in PSMA+PiP tumors and high initial renal uptake that decreases over time. PSMA-620 and PSMA-904 and analogs 60-63 and 65-67 also can act as CI to reduce renal uptake. These compounds can be synthesized analogously to those compounds described in international PCT patent application publication no. WO 2017070482 A2.

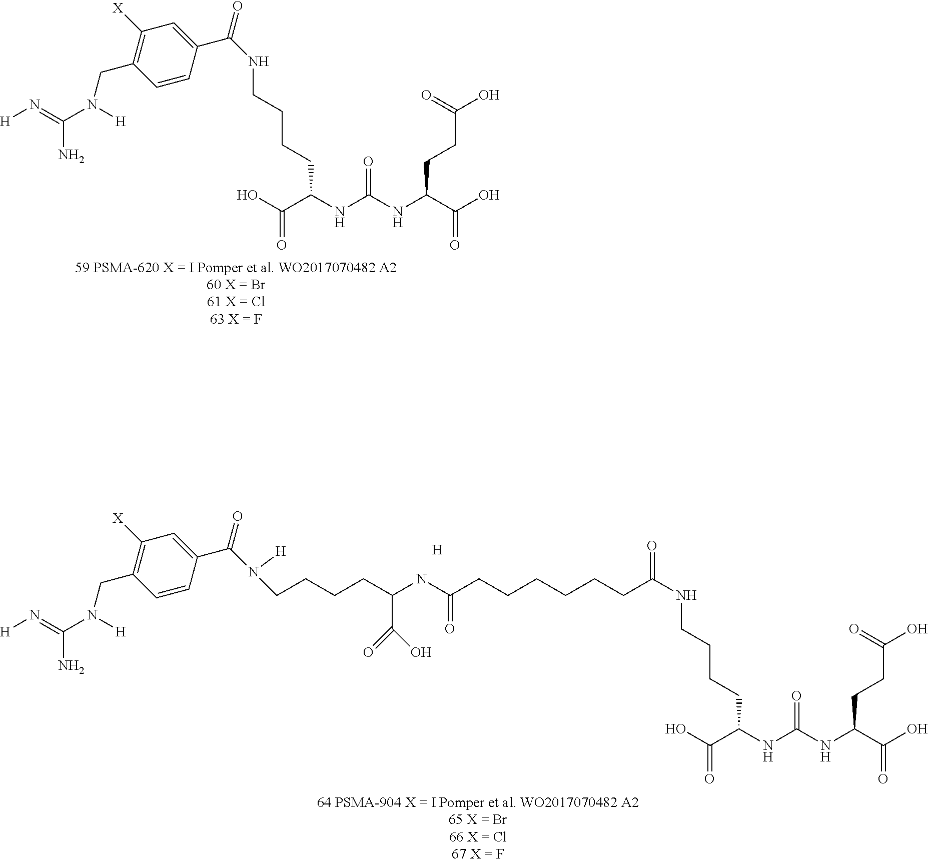

In some embodiments, the competing inhibitor is VK-02-90. In other embodiments, the competing inhibitor is VK-01-45. VK-01-45 and its Lu-177 complex is disclosed in international PCT patent application publication no. WO 2017/165473 A, to Ray et al., which is incorporated herein by reference in its entirety. In yet other embodiments, the competing inhibitor is VK-03-27.

In some embodiments or the presently disclosed methods, the prostate-specific membrane antigen (PSMA)-positive tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof. In particular embodiments, the prostate-specific membrane antigen (PSMA)-positive tumor or cell is primary clear cell renal carcinoma.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

In other embodiments, the presently disclosed method further comprises taking an image. In particular embodiments, the taking of an image is selected from the group consisting of positron emission tomography (PET) and single-photon emission computed tomography (SPECT).

In general, the “effective amount” of an active agent refers to the amount necessary to elicit the desired biological response. As will be appreciated by those of ordinary skill in this art, the effective amount of an agent or device may vary depending on such factors as the desired biological endpoint, the agent to be delivered, the makeup of the pharmaceutical composition, the target tissue, and the like.

The term “combination” is used in its broadest sense and means that a subject is administered at least two agents, more particularly a radiolabeled PSMA inhibitor and a non-radiolabeled PSMA competing inhibitor, and, in some embodiments, at least one other active agent. More particularly, the term “in combination” refers to the concomitant administration of two (or more) active agents for the treatment of a, e.g., single disease state. As used herein, the active agents may be combined and administered in a single dosage form, may be administered as separate dosage forms at the same time, or may be administered as separate dosage forms that are administered alternately or sequentially on the same or separate days. In one embodiment of the presently disclosed subject matter, the active agents are combined and administered in a single dosage form. In another embodiment, the active agents are administered in separate dosage forms (e.g., wherein it is desirable to vary the amount of one but not the other). The single dosage form may include additional active agents for the treatment of the disease state.

In other embodiments, the one or more PSMA-expressing tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof. In some other embodiments, the one or more PSMA-expressing tumor or cell is a prostate tumor or cell.

In other embodiments, the one or more PSMA-expressing tumors or cells is in vitro, in vivo or ex-vivo. In yet other embodiments, the one or more PSMA-expressing tumor or cell is present in a subject.

The subject treated by the presently disclosed methods in their many embodiments is desirably a human subject, although it is to be understood that the methods described herein are effective with respect to all vertebrate species, which are intended to be included in the term “subject.” Accordingly, a “subject” can include a human subject for medical purposes, such as for the treatment of an existing condition or disease or the prophylactic treatment for preventing the onset of a condition or disease, or an animal (non-human) subject for medical, veterinary purposes, or developmental purposes. Suitable animal subjects include mammals including, but not limited to, primates, e.g., humans, monkeys, apes, and the like; bovines, e.g., cattle, oxen, and the like; ovines, e.g., sheep and the like; caprines, e.g., goats and the like; porcines, e.g., pigs, hogs, and the like; equines, e.g., horses, donkeys, zebras, and the like; felines, including wild and domestic cats; canines, including dogs; lagomorphs, including rabbits, hares, and the like; and rodents, including mice, rats, and the like. An animal may be a transgenic animal. In some embodiments, the subject is a human including, but not limited to, fetal, neonatal, infant, juvenile, and adult subjects. Further, a “subject” can include a patient afflicted with or suspected of being afflicted with a condition or disease. Thus, the terms “subject” and “patient” are used interchangeably herein.

B. Kits

In yet other embodiments, the presently disclosed subject matter provides a kit comprising a radiolabeled PSMA inhibitor in combination with a non-radiolabeled PSMA competing inhibitor.

In certain embodiments, the kit provides packaged pharmaceutical compositions comprising a pharmaceutically acceptable carrier and compounds of the invention. In certain embodiments the packaged pharmaceutical composition will comprise the reaction precursors necessary to generate the compound of the invention upon combination with a radio labeled precursor. Other packaged pharmaceutical compositions provided by the present invention further comprise indicia comprising at least one of: instructions for preparing compounds according to the invention from supplied precursors, instructions for using the composition to image cells or tissues expressing PSMA, or instructions for using the composition to image glutamatergic neurotransmission in a patient suffering from a stress-related disorder, or instructions for using the composition to image prostate cancer.

C. Pharmaceutical Compositions and Administration

In another aspect, the present disclosure provides a pharmaceutical composition including a radiolabeled PSMA inhibitor in combination with a non-radiolabeled PSMA competing inhibitor or in combination with one or more additional therapeutic agents in admixture with a pharmaceutically acceptable excipient. One of skill in the art will recognize that the pharmaceutical compositions include the pharmaceutically acceptable salts of the compounds described above. Pharmaceutically acceptable salts are generally well known to those of ordinary skill in the art, and include salts of active compounds which are prepared with relatively nontoxic acids or bases, depending on the particular substituent moieties found on the compounds described herein. When compounds of the present disclosure contain relatively acidic functionalities, base addition salts can be obtained by contacting the neutral form of such compounds with a sufficient amount of the desired base, either neat or in a suitable inert solvent or by ion exchange, whereby one basic counterion (base) in an ionic complex is substituted for another. Examples of pharmaceutically acceptable base addition salts include sodium, potassium, calcium, ammonium, organic amino, or magnesium salt, or a similar salt.

When compounds of the present disclosure contain relatively basic functionalities, acid addition salts can be obtained by contacting the neutral form of such compounds with a sufficient amount of the desired acid, either neat or in a suitable inert solvent or by ion exchange, whereby one acidic counterion (acid) in an ionic complex is substituted for another. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-toluenesulfonic, citric, tartaric, methanesulfonic, and the like. Also included are salts of amino acids such as arginate and the like, and salts of organic acids like glucuronic or galactunoric acids and the like (see, for example, Berge et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

Accordingly, pharmaceutically acceptable salts suitable for use with the presently disclosed subject matter include, by way of example but not limitation, acetate, benzenesulfonate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. Other pharmaceutically acceptable salts may be found in, for example, Remington: The Science and Practice of Pharmacy (20^{th} ed.) Lippincott, Williams & Wilkins (2000). In therapeutic and/or diagnostic applications, the compounds of the disclosure can be formulated for a variety of modes of administration, including systemic and topical or localized administration. Techniques and formulations generally may be found in Remington: The Science and Practice of Pharmacy (20^{th} ed.) Lippincott, Williams & Wilkins (2000).

Depending on the specific conditions being treated, such agents may be formulated into liquid or solid dosage forms and administered systemically or locally. The agents may be delivered, for example, in a timed- or sustained-slow release form as is known to those skilled in the art. Techniques for formulation and administration may be found in Remington: The Science and Practice of Pharmacy (20^{th} ed.) Lippincott, Williams & Wilkins (2000). Suitable routes may include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articular, intra-sternal, intra-synovial, intra-hepatic, intralesional, intracranial, intraperitoneal, intranasal, or intraocular injections or other modes of delivery.

For injection, the agents of the disclosure may be formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation. Such penetrants are generally known in the art.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, the compositions of the present disclosure, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the disclosure to be formulated as tablets, pills, capsules, liquids, gels, syrups, slurries, suspensions and the like, for oral ingestion by a subject (e.g., patient) to be treated.

For nasal or inhalation delivery, the agents of the disclosure also may be formulated by methods known to those of skill in the art, and may include, for example, but not limited to, examples of solubilizing, diluting, or dispersing substances, such as saline; preservatives, such as benzyl alcohol; absorption promoters; and fluorocarbons.

Pharmaceutical compositions suitable for use in the present disclosure include compositions wherein the active ingredients are contained in an effective amount to achieve its intended purpose. Determination of the effective amounts is well within the capability of those skilled in the art, especially in light of the detailed disclosure provided herein. Generally, the compounds according to the disclosure are effective over a wide dosage range. For example, in the treatment of adult humans, dosages from 0.01 to 1000 mg, from 0.5 to 100 mg, from 1 to 50 mg per day, and from 5 to 40 mg per day are examples of dosages that may be used. A non-limiting dosage is 10 to 30 mg per day. The exact dosage will depend upon the route of administration, the form in which the compound is administered, the subject to be treated, the body weight of the subject to be treated, the bioavailability of the compound(s), the adsorption, distribution, metabolism, and excretion (ADME) toxicity of the compound(s), and the preference and experience of the attending physician.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, disintegrating agents may be added, such as the cross-linked polyvinylpyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinylpyrrolidone, carbopol gel, polyethylene glycol (PEG), and/or titanium dioxide, lacquer solutions, and suitable organic solvents or solvent mixtures. Dye-stuffs or pigments may be added to the tablets or dragee coatings for identification or to characterize different combinations of active compound doses.

Pharmaceutical preparations that can be used orally include push-fit capsules made of gelatin, as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols (PEGs). In addition, stabilizers may be added.

### II. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

While the following terms in relation to the presently disclosed compounds are believed to be well understood by one of ordinary skill in the art, the following definitions are set forth to facilitate explanation of the presently disclosed subject matter. These definitions are intended to supplement and illustrate, not preclude, the definitions that would be apparent to one of ordinary skill in the art upon review of the present disclosure.

The terms substituted, whether preceded by the term “optionally” or not, and substituent, as used herein, refer to the ability, as appreciated by one skilled in this art, to change one functional group for another functional group on a molecule, provided that the valency of all atoms is maintained. When more than one position in any given structure may be substituted with more than one substituent selected from a specified group, the substituent may be either the same or different at every position. The substituents also may be further substituted (e.g., an aryl group substituent may have another substituent off it, such as another aryl group, which is further substituted at one or more positions).

Where substituent groups or linking groups are specified by their conventional chemical formulae, written from left to right, they equally encompass the chemically identical substituents that would result from writing the structure from right to left, e.g., —CH₂O— is equivalent to —OCH₂—; —C(═O)O— is equivalent to —OC(═O)—; —OC(═O)NR— is equivalent to —NRC(═O)O—, and the like.

When the term “independently selected” is used, the substituents being referred to (e.g., R groups, such as groups R₁, R₂, and the like, or variables, such as “m” and “n”), can be identical or different. For example, both R₁ and R₂ can be substituted alkyls, or R₁ can be hydrogen and R₂ can be a substituted alkyl, and the like.

The terms “a,” “an,” or “a(n),” when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

A named “R” or group will generally have the structure that is recognized in the art as corresponding to a group having that name, unless specified otherwise herein. For the purposes of illustration, certain representative “R” groups as set forth above are defined below.

Descriptions of compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

Unless otherwise explicitly defined, a “substituent group,” as used herein, includes a functional group selected from one or more of the following moieties, which are defined herein:

The term hydrocarbon, as used herein, refers to any chemical group comprising hydrogen and carbon. The hydrocarbon may be substituted or unsubstituted. As would be known to one skilled in this art, all valencies must be satisfied in making any substitutions. The hydrocarbon may be unsaturated, saturated, branched, unbranched, cyclic, polycyclic, or heterocyclic. Illustrative hydrocarbons are further defined herein below and include, for example, methyl, ethyl, n-propyl, isopropyl, cyclopropyl, allyl, vinyl, n-butyl, tert-butyl, ethynyl, cyclohexyl, and the like.

The term “alkyl,” by itself or as part of another substituent, means, unless otherwise stated, a straight (i.e., unbranched) or branched chain, acyclic or cyclic hydrocarbon group, or combination thereof, which may be fully saturated, mono- or polyunsaturated and can include di- and multivalent groups, having the number of carbon atoms designated (i.e., C₁-C₁₀ means one to ten carbons, including 1, 2, 3, 4, 5, 6, 7, 8, 9, and 10 carbons). In particular embodiments, the term “alkyl” refers to C₁₋₂₀ inclusive, including 1, 2, 3, 4, 5, 6, 7, 8, 9, 10, 11, 12, 13, 14, 15, 16, 17, 18, 19, and 20 carbons, linear (i.e., “straight-chain”), branched, or cyclic, saturated or at least partially and in some cases fully unsaturated (i.e., alkenyl and alkynyl) hydrocarbon radicals derived from a hydrocarbon moiety containing between one and twenty carbon atoms by removal of a single hydrogen atom.

Representative saturated hydrocarbon groups include, but are not limited to, methyl, ethyl, n-propyl, isopropyl, n-butyl, isobutyl, sec-butyl, tert-butyl, n-pentyl, sec-pentyl, isopentyl, neopentyl, n-hexyl, sec-hexyl, n-heptyl, n-octyl, n-decyl, n-undecyl, dodecyl, cyclohexyl, (cyclohexyl)methyl, cyclopropylmethyl, and homologs and isomers thereof “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C₁₋₈ straight-chain alkyls. In other embodiments, “alkyl” refers, in particular, to C₁₋₈ branched-chain alkyls.

Alkyl groups can optionally be substituted (a “substituted alkyl”) with one or more alkyl group substituents, which can be the same or different. The term “alkyl group substituent” includes but is not limited to alkyl, substituted alkyl, halo, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

The term “heteroalkyl,” by itself or in combination with another term, means, unless otherwise stated, a stable straight or branched chain, or cyclic hydrocarbon group, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂₅—S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two or three heteroatoms may be consecutive, such as, for example, —CH₂—NH—OCH₃ and —CH₂—O—Si(CH₃)₃.

As described above, heteroalkyl groups, as used herein, include those groups that are attached to the remainder of the molecule through a heteroatom, such as —C(O)NR′, —NR′R″, —OR′, —SR, —S(O)R, and/or —S(O₂)R′. Where “heteroalkyl” is recited, followed by recitations of specific heteroalkyl groups, such as —NR′R or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. The cycloalkyl group can be optionally partially unsaturated. The cycloalkyl group also can be optionally substituted with an alkyl group substituent as defined herein, oxo, and/or alkylene. There can be optionally inserted along the cyclic alkyl chain one or more oxygen, sulfur or substituted or unsubstituted nitrogen atoms, wherein the nitrogen substituent is hydrogen, unsubstituted alkyl, substituted alkyl, aryl, or substituted aryl, thus providing a heterocyclic group. Representative monocyclic cycloalkyl rings include cyclopentyl, cyclohexyl, and cycloheptyl. Multicyclic cycloalkyl rings include adamantyl, octahydronaphthyl, decalin, camphor, camphane, and noradamantyl, and fused ring systems, such as dihydro- and tetrahydronaphthalene, and the like.

The term “cycloalkylalkyl,” as used herein, refers to a cycloalkyl group as defined hereinabove, which is attached to the parent molecular moiety through an alkyl group, also as defined above. Examples of cycloalkylalkyl groups include cyclopropylmethyl and cyclopentylethyl.

The terms “cycloheteroalkyl” or “heterocycloalkyl” refer to a non-aromatic ring system, unsaturated or partially unsaturated ring system, such as a 3- to 10-member substituted or unsubstituted cycloalkyl ring system, including one or more heteroatoms, which can be the same or different, and are selected from the group consisting of nitrogen (N), oxygen (O), sulfur (S), phosphorus (P), and silicon (Si), and optionally can include one or more double bonds.

The cycloheteroalkyl ring can be optionally fused to or otherwise attached to other cycloheteroalkyl rings and/or non-aromatic hydrocarbon rings. Heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized. In certain embodiments, the term heterocylic refers to a non-aromatic 5-, 6-, or 7-membered ring or a polycyclic group wherein at least one ring atom is a heteroatom selected from O, S, and N (wherein the nitrogen and sulfur heteroatoms may be optionally oxidized), including, but not limited to, a bi- or tri-cyclic group, comprising fused six-membered rings having between one and three heteroatoms independently selected from the oxygen, sulfur, and nitrogen, wherein (i) each 5-membered ring has 0 to 2 double bonds, each 6-membered ring has 0 to 2 double bonds, and each 7-membered ring has 0 to 3 double bonds, (ii) the nitrogen and sulfur heteroatoms may be optionally oxidized, (iii) the nitrogen heteroatom may optionally be quaternized, and (iv) any of the above heterocyclic rings may be fused to an aryl or heteroaryl ring. Representative cycloheteroalkyl ring systems include, but are not limited to pyrrolidinyl, pyrrolinyl, imidazolidinyl, imidazolinyl, pyrazolidinyl, pyrazolinyl, piperidyl, piperazinyl, indolinyl, quinuclidinyl, morpholinyl, thiomorpholinyl, thiadiazinanyl, tetrahydrofuranyl, and the like.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively.

An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl.”

More particularly, the term “alkenyl” as used herein refers to a monovalent group derived from a C₁₋₂₀ inclusive straight or branched hydrocarbon moiety having at least one carbon-carbon double bond by the removal of a single hydrogen molecule. Alkenyl groups include, for example, ethenyl (i.e., vinyl), propenyl, butenyl, 1-methyl-2-buten-1-yl, pentenyl, hexenyl, octenyl, allenyl, and butadienyl.

The term “cycloalkenyl” as used herein refers to a cyclic hydrocarbon containing at least one carbon-carbon double bond. Examples of cycloalkenyl groups include cyclopropenyl, cyclobutenyl, cyclopentenyl, cyclopentadiene, cyclohexenyl, 1,3-cyclohexadiene, cycloheptenyl, cycloheptatrienyl, and cyclooctenyl.

The term “alkynyl” as used herein refers to a monovalent group derived from a straight or branched C₁₋₂₀ hydrocarbon of a designed number of carbon atoms containing at least one carbon-carbon triple bond. Examples of “alkynyl” include ethynyl, 2-propynyl (propargyl), 1-propynyl, pentynyl, hexynyl, and heptynyl groups, and the like.

The term “alkylene” by itself or a part of another substituent refers to a straight or branched bivalent aliphatic hydrocarbon group derived from an alkyl group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀); CH═CH—CH═CH—; CH═CH—CH₂—; —CH₂CH₂CH₂CH₂—, —CH₂CH═CHCH₂—, —CH₂CsCCH₂—, —CH₂CH₂CH(CH₂CH₂CH₃)CH₂—, —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons. Typically, an alkyl (or alkylene) group will have from 1 to 24 carbon atoms, with those groups having 10 or fewer carbon atoms being some embodiments of the present disclosure. A “lower alkyl” or “lower alkylene” is a shorter chain alkyl or alkylene group, generally having eight or fewer carbon atoms.

The term “heteroalkylene” by itself or as part of another substituent means a divalent group derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms also can occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—.

The term “aryl” means, unless otherwise stated, an aromatic hydrocarbon substituent that can be a single ring or multiple rings (such as from 1 to 3 rings), which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent forms of aryl and heteroaryl, respectively.

For brevity, the term “aryl” when used in combination with other terms (e.g., aryloxy, arylthioxy, arylalkyl) includes both aryl and heteroaryl rings as defined above. Thus, the terms “arylalkyl” and “heteroarylalkyl” are meant to include those groups in which an aryl or heteroaryl group is attached to an alkyl group (e.g., benzyl, phenethyl, pyridylmethyl, furylmethyl, and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover only aryls substituted with one or more halogens.

Where a heteroalkyl, heterocycloalkyl, or heteroaryl includes a specific number of members (e.g. “3 to 7 membered”), the term “member” refers to a carbon or heteroatom.

Further, a structure represented generally by the formula:

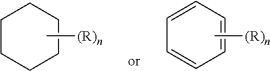

as used herein refers to a ring structure, for example, but not limited to a 3-carbon, a 4-carbon, a 5-carbon, a 6-carbon, a 7-carbon, and the like, aliphatic and/or aromatic cyclic compound, including a saturated ring structure, a partially saturated ring structure, and an unsaturated ring structure, comprising a substituent R group, wherein the R group can be present or absent, and when present, one or more R groups can each be substituted on one or more available carbon atoms of the ring structure. The presence or absence of the R group and number of R groups is determined by the value of the variable “n,” which is an integer generally having a value ranging from 0 to the number of carbon atoms on the ring available for substitution. Each R group, if more than one, is substituted on an available carbon of the ring structure rather than on another R group. For example, the structure above where n is 0 to 2 would comprise compound groups including, but not limited to:

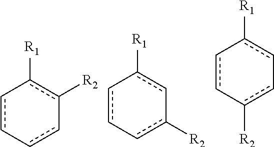

and the like.

A dashed line representing a bond in a cyclic ring structure indicates that the bond can be either present or absent in the ring. That is, a dashed line representing a bond in a cyclic ring structure indicates that the ring structure is selected from the group consisting of a saturated ring structure, a partially saturated ring structure, and an unsaturated ring structure.

The symbol [symbol] denotes the point of attachment of a moiety to the remainder of the molecule.

When a named atom of an aromatic ring or a heterocyclic aromatic ring is defined as being “absent,” the named atom is replaced by a direct bond.

Each of above terms (e.g., “alkyl,” “heteroalkyl,” “cycloalkyl, and “heterocycloalkyl”, “aryl,” “heteroaryl,” “phosphonate,” and “sulfonate” as well as their divalent derivatives) are meant to include both substituted and unsubstituted forms of the indicated group. Optional substituents for each type of group are provided below.

Substituents for alkyl, heteroalkyl, cycloalkyl, heterocycloalkyl monovalent and divalent derivative groups (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″)═NR′″, —S(O)R′, —S(O)₂R′, —S(O)₂NR′R″, —NRSO₂R′, —CN and —NO₂ in a number ranging from zero to (2m′+1), where m′ is the total number of carbon atoms in such groups. R′, R″, R″ and R′″ each may independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. As used herein, an “alkoxy” group is an alkyl attached to the remainder of the molecule through a divalent oxygen. When a compound of the disclosure includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl groups above, exemplary substituents for aryl and heteroaryl groups (as well as their divalent derivatives) are varied and are selected from, for example: halogen, —OR′, —NR′R″, —SR′, —SiR′R″R′″, —OC(O)R′, —C(O)R′, —CO₂R′, —C(O)NR′R″, —OC(O)NR′R″, —NR″C(O)R′, —NR′—C(O)NR″R′″, —NR″C(O)OR′, —NR—C(NR′R″R′″)═NR″″, —NR—C(NR′R″)═NR′″—S(O)R′, —S(O)₂R′, —S(O)₂NR′R″, —NRSO₂R′, —CN and —NO₂, —R′, —N₃, —CH(Ph)₂, fluoro(C₁-C₄)alkoxo, and fluoro(C₁-C₄)alkyl, in a number ranging from zero to the total number of open valences on aromatic ring system; and where R′, R″, R′″ and R″″ may be independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}-U-, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ-B-, wherein A and B are independently —CRR′—, —O—, —NR—, —S—, —S(O)—, —S(O)₂—, —S(O)₂NR′— or a single bond, and r is an integer of from 1 to 4.

One of the single bonds of the new ring so formed may optionally be replaced with a double bond. Alternatively, two of the substituents on adjacent atoms of aryl or heteroaryl ring may optionally be replaced with a substituent of the formula —(CRR′)ₛ—X′—(C″R′″)_{d}—, where s and d are independently integers of from 0 to 3, and X′ is —O—, —NR′—, —S—, —S(O)—, —S(O)₂—, or —S(O)₂NR′—. The substituents R, R′, R″ and R′″ may be independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “acyl” refers to an organic acid group wherein the —OH of the carboxyl group has been replaced with another substituent and has the general formula RC(═O)—, wherein R is an alkyl, alkenyl, alkynyl, aryl, carbocylic, heterocyclic, or aromatic heterocyclic group as defined herein). As such, the term “acyl” specifically includes arylacyl groups, such as a 2-(furan-2-yl)acetyl)- and a 2-phenylacetyl group. Specific examples of acyl groups include acetyl and benzoyl. Acyl groups also are intended to include amides, —RC(═O)NR′, esters, —RC(═O)OR′, ketones, —RC(═O)R′, and aldehydes, —RC(═O)H.

The terms “alkoxyl” or “alkoxy” are used interchangeably herein and refer to a saturated (i.e., alkyl-O—) or unsaturated (i.e., alkenyl-O— and alkynyl-O—) group attached to the parent molecular moiety through an oxygen atom, wherein the terms “alkyl,” “alkenyl,” and “alkynyl” are as previously described and can include C₁₋₂₀ inclusive, linear, branched, or cyclic, saturated or unsaturated oxo-hydrocarbon chains, including, for example, methoxyl, ethoxyl, propoxyl, isopropoxyl, n-butoxyl, sec-butoxyl, tert-butoxyl, and n-pentoxyl, neopentoxyl, n-hexoxyl, and the like.

The term “alkoxyalkyl” as used herein refers to an alkyl-O-alkyl ether, for example, a methoxyethyl or an ethoxymethyl group.

“Aryloxyl” refers to an aryl-O— group wherein the aryl group is as previously described, including a substituted aryl. The term “aryloxyl” as used herein can refer to phenyloxyl or hexyloxyl, and alkyl, substituted alkyl, halo, or alkoxyl substituted phenyloxyl or hexyloxyl.

“Aralkyl” refers to an aryl-alkyl-group wherein aryl and alkyl are as previously described, and included substituted aryl and substituted alkyl. Exemplary aralkyl groups include benzyl, phenylethyl, and naphthylmethyl.

“Aralkyloxyl” refers to an aralkyl-O— group wherein the aralkyl group is as previously described. An exemplary aralkyloxyl group is benzyloxyl, i.e., C₆H₅—CH₂—O—. An aralkyloxyl group can optionally be substituted.

“Alkoxycarbonyl” refers to an alkyl-O—C(═O)— group. Exemplary alkoxycarbonyl groups include methoxycarbonyl, ethoxycarbonyl, butyloxycarbonyl, and tert-butyloxycarbonyl.

“Aryloxycarbonyl” refers to an aryl-O—C(═O)— group. Exemplary aryloxycarbonyl groups include phenoxy- and naphthoxy-carbonyl.

“Aralkoxycarbonyl” refers to an aralkyl-O—C(═O)— group. An exemplary aralkoxycarbonyl group is benzyloxycarbonyl.

“Carbamoyl” refers to an amide group of the formula —C(═O)NH₂. “Alkylcarbamoyl” refers to a R′RN—C(═O)— group wherein one of R and R′ is hydrogen and the other of R and R′ is alkyl and/or substituted alkyl as previously described. “Dialkylcarbamoyl” refers to a R′RN—C(═O)— group wherein each of R and R′ is independently alkyl and/or substituted alkyl as previously described.

The term carbonyldioxyl, as used herein, refers to a carbonate group of the formula —O—C(═O)—OR.

“Acyloxyl” refers to an acyl-O— group wherein acyl is as previously described. The term “amino” refers to the —NH₂ group and also refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

An “aminoalkyl” as used herein refers to an amino group covalently bound to an alkylene linker. More particularly, the terms alkylamino, dialkylamino, and trialkylamino as used herein refer to one, two, or three, respectively, alkyl groups, as previously defined, attached to the parent molecular moiety through a nitrogen atom. The term alkylamino refers to a group having the structure —NHR′ wherein R′ is an alkyl group, as previously defined; whereas the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R″ are each independently selected from the group consisting of alkyl groups. Additionally, R′, R″, and/or R″ taken together may optionally be —(CH₂)ₖ— where k is an integer from 2 to 6. Examples include, but are not limited to, methylamino, dimethylamino, ethylamino, diethylamino, diethylaminocarbonyl, methylethylamino, isopropylamino, piperidino, trimethylamino, and propylamino.

The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

The terms alkylthioether and thioalkoxyl refer to a saturated (i.e., alkyl-S—) or unsaturated (i.e., alkenyl-S— and alkynyl-S—) group attached to the parent molecular moiety through a sulfur atom. Examples of thioalkoxyl moieties include, but are not limited to, methylthio, ethylthio, propylthio, isopropylthio, n-butylthio, and the like.

“Acylamino” refers to an acyl-NH— group wherein acyl is as previously described. “Aroylamino” refers to an aroyl-NH— group wherein aroyl is as previously described.

The term “carbonyl” refers to the —C(═O)— group, and can include an aldehyde group represented by the general formula R—C(═O)H.

The term “carboxyl” refers to the —COOH group. Such groups also are referred to herein as a “carboxylic acid” moiety.

The terms “halo,” “halide,” or “halogen” as used herein refer to fluoro, chloro, bromo, and iodo groups. Additionally, terms such as “haloalkyl,” are meant to include monohaloalkyl and polyhaloalkyl. For example, the term “halo(C₁-C₄)alkyl” is mean to include, but not be limited to, trifluoromethyl, 2,2,2-trifluoroethyl, 4-chlorobutyl, 3-bromopropyl, and the like.

The term “hydroxyl” refers to the —OH group.

The term “hydroxyalkyl” refers to an alkyl group substituted with an —OH group.

The term “mercapto” refers to the —SH group.

The term “oxo” as used herein means an oxygen atom that is double bonded to a carbon atom or to another element.

The term “nitro” refers to the —NO₂ group.

The term “thio” refers to a compound described previously herein wherein a carbon or oxygen atom is replaced by a sulfur atom.

The term “sulfate” refers to the —SO₄ group.

The term thiohydroxyl or thiol, as used herein, refers to a group of the formula —SH.

More particularly, the term “sulfide” refers to compound having a group of the formula —SR.

The term “sulfone” refers to compound having a sulfonyl group —S(O₂)R.

The term “sulfoxide” refers to a compound having a sulfinyl group —S(O)R

The term ureido refers to a urea group of the formula —NH—CO—NH₂.

The term “protecting group” in reference to the presently disclosed compounds refers to a chemical substituent which can be selectively removed by readily available reagents which do not attack the regenerated functional group or other functional groups in the molecule. Suitable protecting groups are known in the art and continue to be developed. Suitable protecting groups may be found, for example in Wutz et al. (“Greene's Protective Groups in Organic Synthesis, Fourth Edition,” Wiley-Interscience, 2007). Protecting groups for protection of the carboxyl group, as described by Wutz et al. (pages 533-643), are used in certain embodiments. In some embodiments, the protecting group is removable by treatment with acid. Representative examples of protecting groups include, but are not limited to, benzyl, p-methoxybenzyl (PMB), tertiary butyl (t-Bu), methoxymethyl (MOM), methoxyethoxymethyl (MEM), methylthiomethyl (MTM), tetrahydropyranyl (THP), tetrahydrofuranyl (THF), benzyloxymethyl (BOM), trimethylsilyl (TMS), triethylsilyl (TES), t-butyldimethylsilyl (TBDMS), and triphenylmethyl (trityl, Tr). Persons skilled in the art will recognize appropriate situations in which protecting groups are required and will be able to select an appropriate protecting group for use in a particular circumstance.

Throughout the specification and claims, a given chemical formula or name shall encompass all tautomers, congeners, and optical- and stereoisomers, as well as racemic mixtures where such isomers and mixtures exist.

Certain compounds of the present disclosure may possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as D- or L- for amino acids, and individual isomers are encompassed within the scope of the present disclosure. The compounds of the present disclosure do not include those which are known in art to be too unstable to synthesize and/or isolate. The present disclosure is meant to include compounds in racemic, scalemic, and optically pure forms. Optically active (R)- and (S)-, or D- and L-isomers may be prepared using chiral synthons or chiral reagents, or resolved using conventional techniques. When the compounds described herein contain olefenic bonds or other centers of geometric asymmetry, and unless specified otherwise, it is intended that the compounds include both E and Z geometric isomers.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

It will be apparent to one skilled in the art that certain compounds of this disclosure may exist in tautomeric forms, all such tautomeric forms of the compounds being within the scope of the disclosure. The term “tautomer,” as used herein, refers to one of two or more structural isomers which exist in equilibrium and which are readily converted from one isomeric form to another.

Unless otherwise stated, structures depicted herein are also meant to include compounds which differ only in the presence of one or more isotopically enriched atoms. For example, compounds having the present structures with the replacement of a hydrogen by a deuterium or tritium, or the replacement of a carbon by ¹³C- or ¹⁴C-enriched carbon are within the scope of this disclosure.

The compounds of the present disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present disclosure, whether radioactive or not, are encompassed within the scope of the present disclosure.

The compounds of the present disclosure may exist as salts. The present disclosure includes such salts. Examples of applicable salt forms include hydrochlorides, hydrobromides, sulfates, methanesulfonates, nitrates, maleates, acetates, citrates, fumarates, tartrates (e.g. (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent or by ion exchange. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents.

Certain compounds of the present disclosure can exist in unsolvated forms as well as solvated forms, including hydrated forms. In general, the solvated forms are equivalent to unsolvated forms and are encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

In addition to salt forms, the present disclosure provides compounds, which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### YC-I-27 (DCIBzL) as a Competing Inhibitor

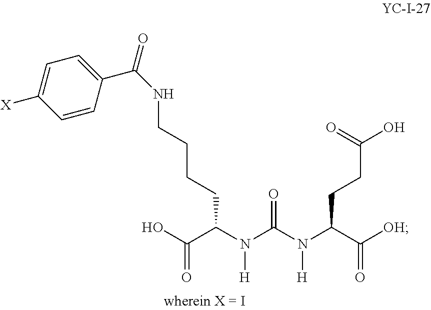

YC-I-27 (also referred to herein as DCIBzL) was first reported by in 2008 (Y. Chen et al. J. Med. Chem. 51:7933-7933, 2008) as an imaging agent and subsequently disclosed in international PCT patent application publication no. WO2008058192 A2, to Babich et al., which is incorporated herein by reference in its entirety. It is a very strong PSMA inhibitor having a Kᵢ=0.01 nM. When prepared in 1-125 labeled form it exhibited high and prolonged uptake in PSMA positive PiP tumors. YC-I-27 also demonstrated very high uptake and retention in kidneys (>100% ID/g out to two days post-injection). The strong binding of YC-I-27 to PSMA is likely due to the localization of the iodobenzoyl portion of YC-I-27 into a hydrophobic pocket adjacent to the PSMA binding site as determined by X-ray crystallography (C. Barinka et al. J. Med. Chem. 51:7737-7743, 2008).

Due to its very high and prolonged uptake in the kidneys, YC-I-27 was an initial choice to use as a competing PSMA binding agent to reduce the renal uptake of radiolabeled PSMA inhibitors. Tables 1, 2, and 3 demonstrate the ability of a co-injection of 0.5 nmoles of non-radioactive YC-I-27 with various radiohalogenated PSMA inhibitors, including [^{131/125}I] YC-I-27 (self competition), [¹²⁵I]HS-549 (international PCT patent application publication no. WO 2017/070482 A2, to Pomper et al., which is incorporated herein by reference in its entirety), [¹²⁵I]VK-02-90, [¹²⁵I]VK-02-90-Lu, and [²¹¹At]VK-02-90-Lu (disclosed in U.S. provisional patent application No. 62/626,993, to Pomper et al., filed Feb. 6, 2018, which is incorporated herein by reference in its entirety) to reduce the uptake in kidneys and, to a lesser extent, in the salivary and lacrimal glands, especially early (1 hr) after injection compared to injection without competing inhibitor.

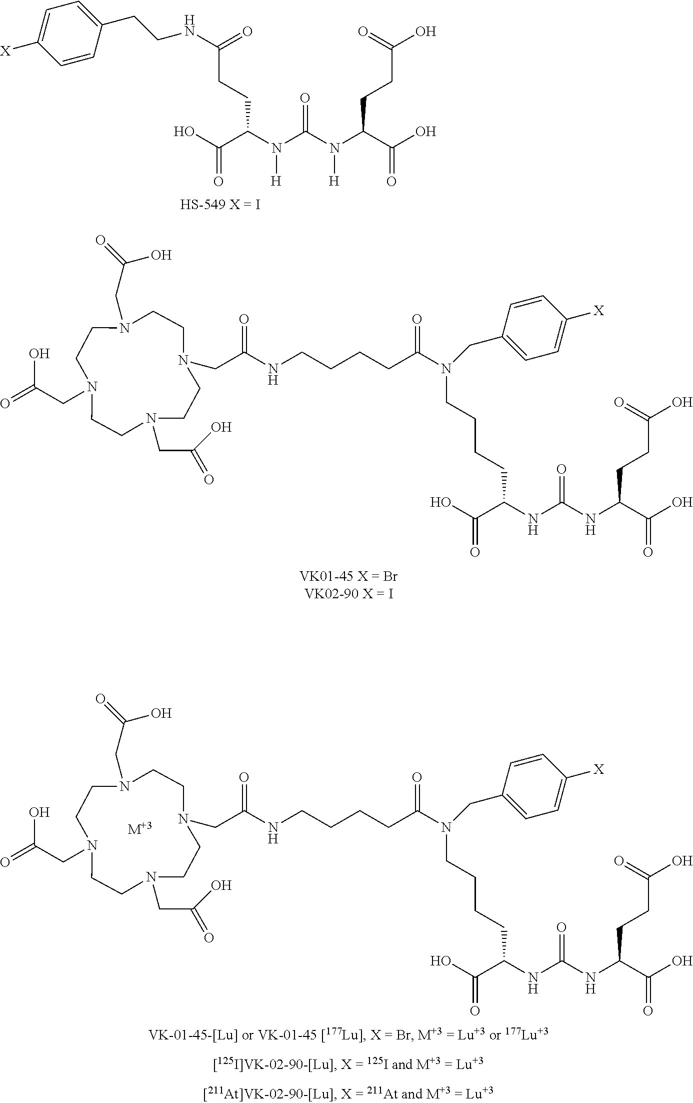

TABLE 1
One-Hour Biodistribution (% injected dose/gram) of Radiohalogenated PSMA
inhibitors in mice bearing PSMA+ PC-3 PiP and PSMA− PC-3 flu tumors without and with co-
injection of 0.5 nmoles of competing inhibitor YC-I-27.

| | [^{131/125}I]YC-I-27 | | [¹²⁵I]HS-549 | | [¹²⁵I]VK-02-90 | | [¹²⁵I]VK-02-90-Lu | | [²¹¹At]VK-02-90-Lu | |
|---|---|---|---|---|---|---|---|---|---|---|
| | | 0.5 nmole | | 0.5 nmole | | 0.5 nmole | | 0.5 nmole | | 0.5 nmole |
| | No CI | YC-I-27 | No CI | YC-I-27 | No CI | YC-I-27 | No CI | YC-I-27 | No CI | YC-I-27 |
| Blood | 1.6 ± 0.7 | 1.4 ± 0.08 | 5.9 ± 1.0 | 2.6 ± 0.6 | 1.1 ± 0.3 | 0.45 ± 0.06 | 1.1 ± 0.1 | 1.1 ± 0.1 | 0.8 ± 0.2 | 0.5 ± 0.1 |
| stomach | 0.9 ± 0.16 | 1.1 ± 0.15 | 0.9 ± 0.2 | 0.8 ± 0.3 | 1.1 ± 0.3 | 0.33 ± 0.07 | 1.7 ± 0.3 | 1.8 ± 0.2 | 0.4 ± 0.1 | 0.4 ± 0.2 |
| spleen | 21.2 ± 3.6 | 7.9 ± 1.5 | 6.8 ± 3.4 | 0.8 ± 0.2 | 40 ± 16 | 0.8 ± 0.14 | 4.3 ± 0.9 | 0.8 ± 0.2 | 2.5 ± 0.9 | 0.3 ± 0.4 |
| thyroid | 0.45 ± 0.14 | 1.0 ± 0.42 | 1.2 ± 0.4 | 5.6 ± 9.2 | NC | 0.5 ± 0.3 | ^{†}NC | NC | NC | NC |
| Salivary | NC | 2.47 ± 0.64 | NC | 1.0 ± 0.2 | 5.1 ± 3.4 | 0.46 ± 0.06 | 2.3 ± 0.5 | 3.2 ± 0.7 | 0.5 ± 0.2 | 0.4 ± 0.2 |
| gland | | | | | | | | | | |
| Lacrimal | NC | 3.5 ± 0.95 | NC | 1.1 ± 0.4 | 17.4 ± 6.0 | 0.75 ± 0.33 | 2.1 ± 0.9 | 0.5 ± 0.1 | 0.8 ± 0.9 | 0 |
| gland | | | | | | | | | | |
| kidneys | 118 ± 17 | 46 ± 2.8 | 106 ± 19 | 12.1 ± 1.9 | 194 ± 25 | 10.0 ± 1.1 | 129 ± 15 | 4.2 ± 1.3 | 90 ± 43 | 2.2 ± 0.6 |
| PiP | 13.1 ± 5.5 | 44.5 ± 5.3 | 78 ± 19 | 31 ± 4.2 | 58 ± 14 | 35 ± 10 | 26 ± 3 | 42 ± 4 | 31 ± 5 | 25 ± 4 |
| tumor | | | | | | | | | | |
| Flu | 0.6 ± 0.1 | 1.3 ± 0.5 | 2.2 ± 0.5 | 1.0 ± 0.45 | 1.4 ± 0.6 | 0.37 ± 0.05 | 1.0 ± 0.2 | 1.0 ± 0.3 | 0.4 ± 0.2 | 0.4 ± 0.1 |
| tumor | | | | | | | | | | |

^{†}NC = not collected.

TABLE 2
Four-Hour Biodistribution (% injected dose/gram) of Radiohalogenated PSMA inhibitors
in mice bearing PSMA+ PC-3 PiP and PSMA− PC-3 flu tumors without and with co-injection of
0.5 nmoles of competing inhibitor YC-I-27.

| | [^{131/125}I] YC-I-27 | | [^{131/125}I]HS-549 | | [¹²⁵I]VK-02-90 | | [¹²⁵I]VK-02-90-Lu | | [²¹¹At]VK-02-90-Lu | |
|---|---|---|---|---|---|---|---|---|---|---|
| | | 0.5 nmole | | 0.5 nmole ^{a} | | 0.5 nmole | | 0.5 nmole | | 0.5 nmole |
| | No CI | YC-I-27 | No CI | YC-I-27 | No CI | YC-I-27 | No CI | YC-I-27 | No CI | YC-I-27 |
| Blood | 0.6 ± 0.3 | 0.2 ± 0.05 | 2.9 ± 0.3 | 0.8 ± 0.1 | 0.2 ± 0.03 | 0.02 ± 0.004 | 0.3 ± 0.2 | 0.4 ± 0.06 | 0.1 ± 0.03 | 0.2 ± 0.1 |
| stomach | 0.7 ± 0.2 | 0.4 ± 0.3 | 0.5 ± 0.2 | 0.3 ± 0.1 | 0.4 ± 0.2 | 0.2 ± 0.07 | 0.8 ± 0.5 | 1.3 ± 0.2 | 0.3 ± 0.2 | 0.4 ± 0.05 |
| spleen | 18 ± 6 | 5.1 ± 1.7 | 2.7 ± 0.8 | 0.4 ± 0.1 | 4.7 ± 1.2 | 0.2 ± 0.1 | 0.6 ± 0.2 | 0.25 ± 0.07 | 0 | 0.8 ± 0.5 |
| thyroid | 0.1 ± 0.2 | 0.3 ± 0.1 | 0.7 ± 0.3 | 0.3 ± 0.1 | ^{†}NC | 0.14 ± 0.1 | NC | NC | NC | NC |
| Salivary | NC | 0.9 ± 0.2 | NC | 0.4 ± 0.2 | 0.7 ± 0.2 | 0.08 ± 0.04 | 1.8 ± 0.2 | 3.3 ± 0.8 | 0.2 ± 0.1 | 0.3 ± 0.2 |
| gland | | | | | | | | | | |
| Lacrimal | NC | 2.4 ± 0.6 | NC | 0.5 ± 0.3 | 1.9 ± 0.5 | 0.14 ± 0.08 | 0.4 ± 0.2 | 0.2 ± 0.02 | 0.3 ± 1.1 | 0 |
| gland | | | | | | | | | | |
| kidneys | 117 ± 33 | 38 ± 3 | 97 ± 39 | 2.8 ± 0.8 | 162 ± 3.5 | 2.2 ± 0.3 | 9.5 ± 2.5 | 0.9 ± 0.1 | 2.1 ± 0.6 | 0.7 ± 0.1 |
| PiP | 16 ± 3.8 | 58 ± 13 | 75 ± 13 | 39 ± 4 | 38 ± 13 | 35 ± 5.0 | 27 ± 7 | 24 ± 3 | 17 ± 5 | 19 ± 5 |
| tumor | | | | | | | | | | |
| Flu | 0.2 ± 0.1 | 0.3 ± 0.1 | 1.5 ± 0.3 | 0.3 ± 0.15 | 0.3 ± 0.1 | 0.7 ± 0.01 | 0.2 ± 0.1 | 0.3 ± 0.1 | 0.1 ± 0.1 | 0.0 ± 0.04 |
| tumor | | | | | | | | | | |

^{†}NC = not collected;
^{a} = 2 hours

TABLE 3
24-Hour Biodistribution (% injected dose/gram) of Radiohalogenated PSMA inhibitors
in mice bearing PSMA+ PC-3 PiP and PSMA− PC-3 flu tumors without and with co-injection of
0.5 nmoles of competing inhibitor YC-I-27.

| | [^{131/125}I]YC-I-27 | | [^{131/125}I]HS-549 | | [¹²⁵I]VK-02-90 | | [¹²⁵I]VK-02-90-Lu | | [²¹¹At]VK-02-90-Lu | |
|---|---|---|---|---|---|---|---|---|---|---|
| | | 0.5 nmole | | 0.5 nmole | | 0.5 nmole | | 0.5 nmole | | 0.5 nmole |
| | No CI^{b} | YC-I-27 | No CI^{b} | YC-I-27 | No CI | YC-I-27 | No CI | YC-I-27 | No CI | YC-I-27 |
| Blood | 0.16 ± 0.06 | 0.02 ± 0.003 | 1.4 ± 0.4 | 0.6 ± 0.3 | 0.02 ± 0.01 | 0.001 ± 0.001 | 0.01 ± 0.01 | 0.1 ± 0.01 | 0 | 0.03 ± 0.3 |
| stomach | 0.4 ± 0.07 | 015 ± 0.06 | 0.1 ± 0.04 | 0.1 ± 0.05 | 0.05 ± 0.03 | 0.06 ± 0.02 | 0.01 ± 0.04 | 0.04 ± 0.04 | 0 | 0.4 ± 0.7 |
| spleen | 5.2 ± 1.5 | 2.5 ± 0.7 | 0.3 ± 0.1 | 0.1 ± 0.07 | 0.7 ± 0.4 | 0.05 ± 0.04 | 0.1 ± 0.05 | 0.04 ± 0.01 | 0 | 02.5 ± 4.3 |
| thyroid | 0.1 ± 0.1 | 0.2 ± 0.1 | 0.4 ± 0.05 | 0.5 ± 0.8 | ^{†}NC | 0.04 ± 0.08 | NC | NC | NC | NC |
| Salivary | NC | 0.3 ± 0.01 | NC | 0.2 ± 0.1 | 0.3 ± 0.3 | 0.02 ± 0.01 | 0.02 ± 0.08 | 0.02 ± 0.01 | 0 | 0 |
| gland | | | | | | | | | | |
| Lacrimal | NC | 1.0 ± 0.1 | NC | 0.2 ± 0.1 | 0.5 ± 0.2 | 0.02 ± 0.04 | 0.05 ± 0.04 | 0.03 ± 0.02 | 0 | 0 |
| gland | | | | | | | | | | |
| kidneys | 126 ± 28 | 24 ± 3 | 3.1 ± 1.6 | 0.7 ± 0.3 | 31 ± 14 | 0.3 ± 0.03 | 1.3 ± 0.7 | 0.5 ± 0.2 | 0.02 ± 0.02 | 0.4 ± 0.5 |
| PiP | 26 ± 10 | 74 ± 5 | 23 ± 2 | 35 ± 5 | 51 ± 7 | 33 ± 6 | 18 ± 6 | 16 ± 4 | 9.5 ± 1.0 | 18 ± 8 |
| tumor | | | | | | | | | | |
| Flu | 0.6 ± 0.01 | 0.05 ± 0.02 | 0.4 ± 0.2 | 0.2 ± 0.2 | 0.04 ± 0.02 | 0.02 ± 0.01 | 0.05 ± 0.01 | 0.04 ± 0.01 | 0 | 0.2 ± 0.3 |
| tumor | | | | | | | | | | |

^{†}NC = not collected;
^{b}= 21 hours

Table 4 Demonstrates that the reduction in kidney uptake and retention at early time points is CI dose dependent.

TABLE 4
One- and Four-Hour Biodistribution (% injected dose/gram) of [¹²⁵I]VK-02-90-Lu in
mice bearing PSMA+ PC-3 PiP and PSMA− PC-3 flu tumors without and with co-injection of
0.5 nmoles, 1.0 nmole, or 5 nmole of competing inhibitor YC-I-27.

| | One Hour | | | | Four Hour | | | |
|---|---|---|---|---|---|---|---|---|
| | [¹²⁵I]VK-02-90-Lu | | | | [¹²⁵I]VK-02-90-Lu | | | |
| | | 0.5 nmole | 1.0 nmole | 5.0 nmole | | 0.5 nmole | 1.0 nmole | 5.0 nmole |
| | No CI | YC-I-27 | YC-I-27 | YC-I-27 | No CI | YC-I-27 | YC-I-27 | YC-I-27 |
| Blood | 1.1 ± 0.1 | 1.1 ± 0.1 | 1.0 ± 0.1 | 1.1 ± 0.2 | 0.3 ± 0.2 | 0.4 ± 0.06 | 0.4 ± 0.05 | 0.4 ± 0.1 |
| Stomach | 1.7 ± 0.3 | 1.8 ± 0.2 | 1.8 ± 0.3 | 2.2 ± 0.7 | 0.8 ± 0.5 | 1.3 ± 0.2 | 1.2 ± 0.2 | 1.6 ± 0.4 |
| Spleen | 4.3 ± 0.9 | 0.8 ± 0.2 | 0.6 ± 0.1 | 0.7 ± 0.1 | 0.6 ± 0.2 | 0.25 ± 0.07 | 0.2 ± 0.04 | 0.3 ± 0.06 |
| Salivary | 2.3 ± 0.5 | 3.2 ± 0.7 | 2.9 ± 0.4 | 5.1 ± 1.7 | 1.8 ± 0.2 | 3.3 ± 0.8 | 3.2 ± 0.7 | 5.0 ± 1.4 |
| Gland | | | | | | | | |
| Lacrimal | 2.1 ± 0.9 | 0.5 ± 0.1 | 0.6 ± 0.05 | 1.1 ± 0.6 | 0.4 ± 0.2 | 0.4 ± 0.02 | 0.2 ± 0.03 | 0.5 ± 0.5 |
| Gland | | | | | | | | |
| Kidneys | 129 ± 15 | 4.2 ± 1.3 | 2.4 ± 0.8 | 2.3 ± 0.7 | 10 ± 2.5 | 0.9 ± 0.1 | 0.6 ± 0.03 | 0.7 ± 0.1 |
| PiP | 26 ± 3 | 42 ± 4.2 | 29 ± 2 | 20 ± 3 | 27 ± 8 | 24 ± 3 | 24 ± 1 | 14 ± 5 |
| Tumor | | | | | | | | |
| Flu | 1.0 ± 0.2 | 1.0 ± 0.3 | 0.8 ± 0.2 | 0.8 ± 0.2 | 0.25 ± 0.1 | 0.3 ± 0.1 | 0.3 ± 0.1 | 0.3 ± 0.1 |
| Tumor | | | | | | | | |

Compounds similar to YC-I-27 having a 4-substituted, 3-substituted-, or 2-fluoro-4-substituted benzoyl group include compounds 1-23.

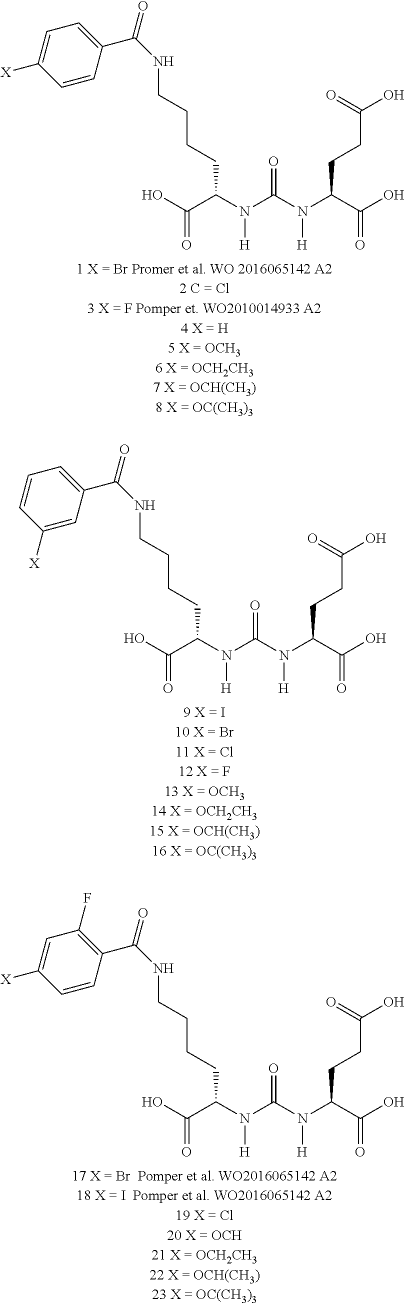

Each of compounds 1-23 can be synthesized from lysine-glutamate-urea-tributylate and commercial substituted benzoic acids as shown immediately hereinbelow.

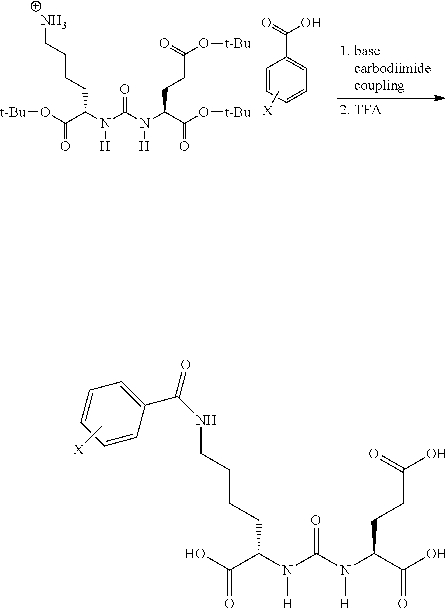

### Example 2

### Reverse Carbamates as Competing Inhibitors

Reverse carbamates compounds 24 and 25 were first reported in 2016 (X. Yang et al. J. Med. Chem. 59: 206-218, 2016 and international PCT patent application publication no. WO2016065145 A2, to Pomper et al., which is incorporated herein by reference in its entirety). They are strong inhibitors of PSMA (Kᵢ=0.2 nM and 0.1 nM respectively). When labeled with ¹⁸F they each demonstrated high uptake in PSMA+PiP tumors and high initial renal uptake that decreases over time. Compounds 24 and 25 and analogs 26-30 are thus candidates for CI to reduce renal uptake. They can be prepared as described in Yang et al. (J. Med. Chem. 59: 206-218, 2016) using appropriately substituted benzoic acids.

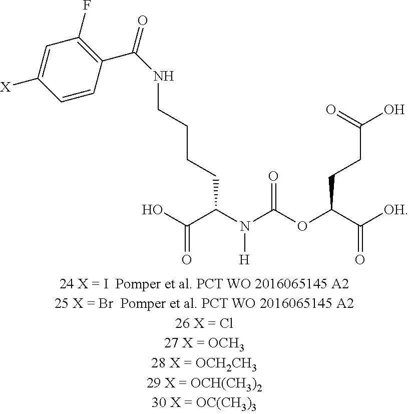

### Example 3

### Halo-nicotimamides as Competing Inhibitors

4- and 5-halo-nicotinamide-lysine-glutamine ureas also are PSMA inhibitors. Compound 31 and compound 38 (DCFPyL) were first reported by the Pomper group (Y. Chen et al. J. Med. Chem. 51: 7933-7943, 2008; Clinical Cancer research 17: 7645-7653, 2011). Each demonstrated high uptake in PSMA+PiP tumors and high initial renal uptake that decreases over time. These compounds and analogs 32-37 also can act as CI to reduce renal uptake. These compounds can be synthesized analogously to those above using the appropriately substituted nicotinic acid.

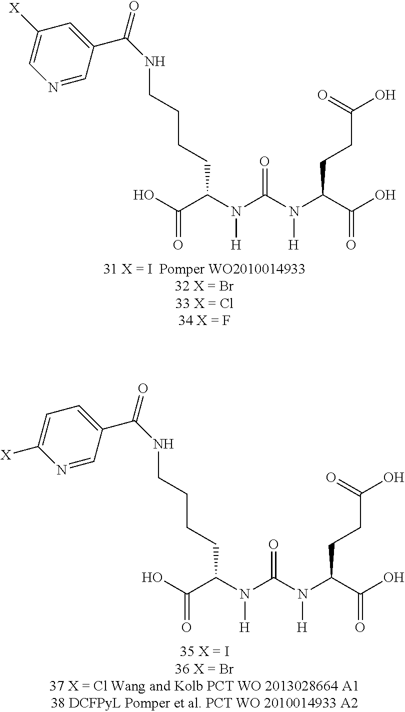

### Example 4

### HS-549 and HS-549 Analogs as Competing Inhibitors

Compound 38 (HS-549) was first reported in international PCT patent application publication no. WO 2017070482 A2, to Pomper et al. In PSMA docking studies it overlaps with YC-I-27 exactly in the PSMA binding pocket and exhibits the same very strong binding (Ki=0.01 nM). Like YC-I-27, its radioiodinated version has high PSMA+Pip tumor uptake and high initial kidney uptake. But, unlike YC-I-27, the kidney clears with time similar to that of the halo-nicotinamide compounds described herein above. Therefore, HS-549 and its analogs 39-58 also can act as CI to reduce renal uptake. Compounds 39-58 can be synthesized as described for HS-549 in international PCT patent application publication no. WO 2017070482 A2 to Pomper et al., using commercial substituted phenethylamines.

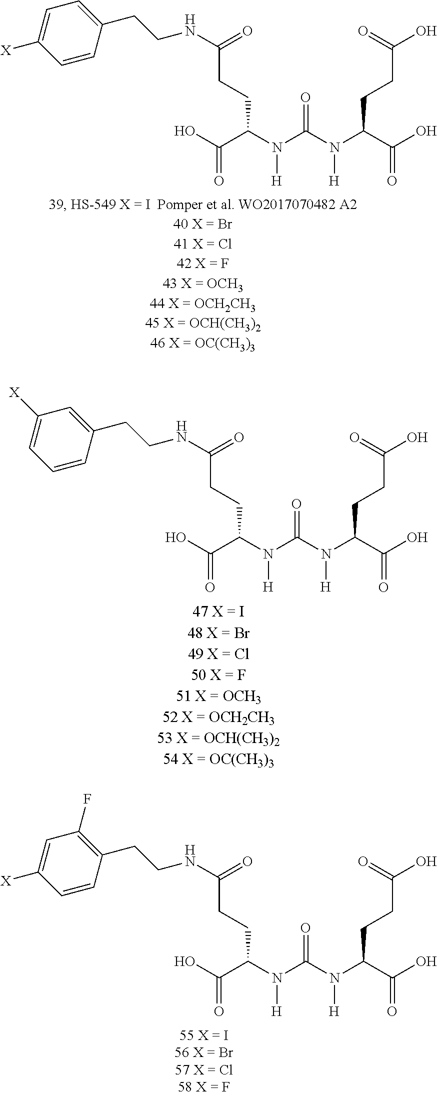

### Example 5

### PSMA-620 and PSMA-904 Analogs as Competing Inhibitors

PSMA-620 and PSMA-904 were reported in international PCT patent application publication no. WO 2017070482 A2, to Pomper et al., which is incorporated herein by reference in its entirety. PSMA-620 and PSMA-904 each demonstrated high uptake in PSMA+PiP tumors and high initial renal uptake that decreases over time. PSMA-620 and PSMA-904 and analogs 60-63 and 65-67 also can act as CI to reduce renal uptake. These compounds can be synthesized analogously to those described in WO 2017070482 A2.

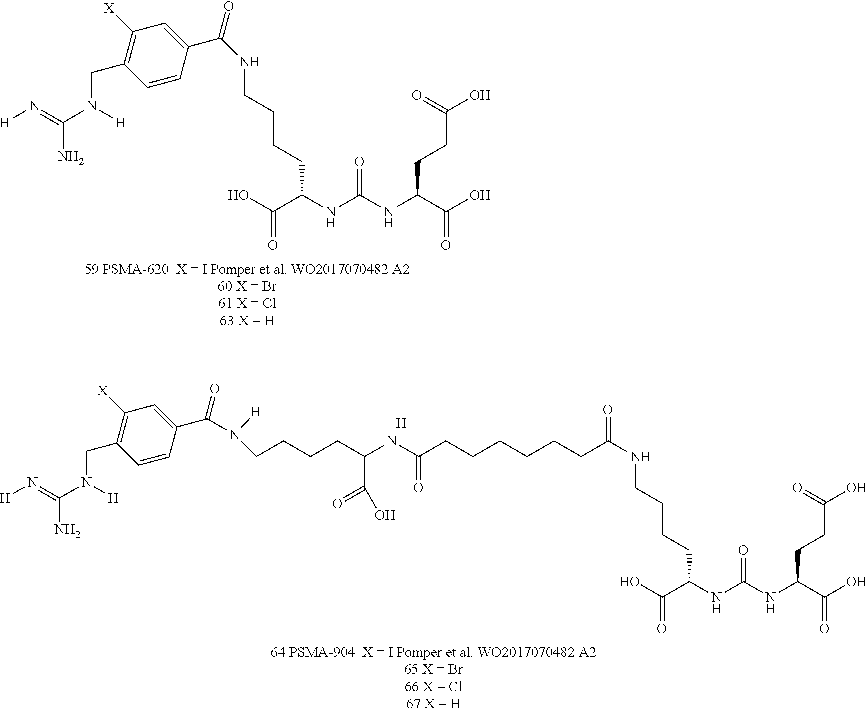

### Example 6

### VK-02-90 as a Competing Inhibitor

As shown in Tables 1, 2, and 3, [¹²⁵I]VK-02-90 exhibits high PSMA+ tumor uptake and high initial kidney uptake which clears over time. Table 5 demonstrates that VK-02-90 is an effective CI for reducing the uptake of [¹²⁵I]VK-02-90-Lu in kidneys, salivary glands, and lacrimal glands at early time points.

TABLE 5
One-and Four-Hour Biodistribution (% injected dose/gram) of [¹²⁵I]VK-
02-90-Lu in mice bearing PSMA+ PC-3 PiP and PSMA− PC-3 flu tumors without
and with co-injection of 0.5 nmoles, 1.0 nmole, or 5 nmole of competing inhibitor
VK-02-90.

| | 2 Hour | | | | 4 Hour | | | |
|---|---|---|---|---|---|---|---|---|
| | [¹²⁵I]VK-02-90-Lu | | | | [¹²⁵I]VK-02-90-Lu | | | |
| | | 0.5 nmole | 1.0 nmole | 5.0 nmole | | 0.5 nmole | 1.0 nmole | 5.0nmole |
| | No CI | VK-02-90 | VK-02-90 | VK-02-90 | No CI | VK-02-90 | VK-02-90 | VK-02-90 |
| Blood | 1.1 ± 0.1 | 0.4 ± 0.1 | 0.4 ± 0.1 | 0.5 ± 0.1 | 0.3 ± 0.2 | 0.02 ± 0.01 | 0.2 ± 0.01 | 0 |
| stomach | 1.7 ± 0.3 | 0.2 ± 0.1 | 0.2 ± 0.04 | 0.2 ± 0.06 | 0.8 ± 0.5 | 0.06 ± 0.01 | 0.1 ± 0.03 | 0.1 ± 0.04 |
| spleen | 4.3 ± 0.9 | 1.1 ± 0.4 | 1.1 ± 0.4 | 0.7 ± 0.2 | 0.6 ± 0.2 | 0.2 ± 0.1 | 0.2 ± 0.1 | 0.15 ± 0.08 |
| Salivary | 2.3 ± 0.5 | 0.2 ± 0.03 | 0.2 ± 0.06 | 0.3 ± 0.1 | 1.8 ± 0.2 | 0.08 ± 0.04 | 0.1 ± 0.02 | 0.05 ± 0.02 |
| gland | | | | | | | | |
| Lacrimal | 2.1 ± 0.9 | 0.4 ± 0.1 | 0.4 ± 0.1 | 0.4 ± 0.03 | 0.4 ± 0.2 | 0.1 ± 0.03 | 0.1 ± 0.05 | 0.1 ± 0.1 |
| gland | | | | | | | | |
| kidneys | 129 ± 15 | 10 ± 2.5 | 6.5 ± 0.9 | 4.2 ± 0.5 | 10 ± 2.5 | 2.1 ± 0.7 | 1.4 ± 0.4 | 0.9 ± 0.3 |
| PiP | 26 ± 3 | 37 ± 7 | 35 ± 9 | 24 ± 4 | 27 ± 8 | 25 ± 2 | 27 ± 4 | 19 ± 2 |
| tumor | | | | | | | | |
| Flu | 1.0 ± 0.2 | 0.6 ± 0.3 | 0.5 ± 0.1 | 0.9 ± 0.3 | 0.2 ± 0.1 | 0.1 ± 0.02 | 0.1 ± 0.03 | 0.1 ± 0.01 |
| tumor | | | | | | | | |

### Example 7

### VK-01-45 as a Competing Inhibitor

VK-01-45 and its Lu-177 complex was reported in international PCT patent application publication no. WO 2017/165473 A, to Ray et al., which is incorporated herein by reference in its entirety. The biodistribution data presented in Table 6 show that the two-hour kidney uptake and retention of radiometal labeled PSMA binding agents [¹⁷⁷Lu]VK-01-45 also can be reduced by co-injection of the non-radioactive metal labeled version [Lu]VK-01-45 (self blocking) with only a modest reduction in PSMA+PiP tumor uptake. This approach can be applied to all compounds in WO 2017/165473 A1, to Ray et al.

TABLE 6
Two-Hour Biodistribution (% injected dose/gram) of
[¹⁷⁷Lu]VK-I-45 in mice bearing PSMA + PC-3 PiP
and PSMA − PC-3 flu tumors without and
with co-injection of 0.1 nmoles, 0.5 nmole, 1.0 nmole, or
5.0 nmole of competing inhibitor [Lu]VK-I-45.

| | 2 Hour [¹⁷⁷Lu]VK-I-45 | | | | |
|---|---|---|---|---|---|
| | | 0.1 nmole | 0.5 nmole | 1.0 nmole | 5.0 nmole |
| | | [Lu]VK- | [Lu]VK- | [Lu]VK- | [Lu]VK- |
| | No CI | I-45 | I-45 | I-45 | I-45 |
| Blood | 0.1 ± 0.04 | 0.12 ± 0.02 | 0.11 ± 0.04 | 0.07 ± 0.02 | 0.8 ± 0.04 |
| Stomach | 0.1 ± 0.04 | 0.2 ± 0.08 | 0.09 ± 0.02 | 0.13 ± 0.08 | 0.2 ± 0.18 |
| Spleen | 0.7 ± 0.2 | 0.65 ± 0.1 | 0.32 ± 0.07 | 0.15 ± 0.02 | 0.14 ± 0.06 |
| Salivary | 0.16 ± 0.07 | 0.16 ± 0.03 | 0.12 ± 0.02 | 0.07 ± 0.01 | 0.11 ± 0.04 |
| Gland | | | | | |
| Kidneys | 35 ± 17 | 17 ± 11 | 4.8 ± 1.4 | 1.75 ± 0.32 | 1.35 ± 0.44 |
| PiP | 39 ± 14 | 38 ± 4 | 35 ± 8 | 20 ± 1.5 | 17.3 ± 1.9 |
| Tumor | | | | | |
| Flu | 0.3 ± 0.06 | 0.4 ± 0.07 | 0.35 ± 0.07 | 0.2 ± 0.03 | 0.36 ± 0.14 |
| Tumor | | | | | |

### Example 8

### VK-03-27 as a Competing Inhibitor

VK-03-27 is a new DOTA-Ga analog of VK-I-45. Its synthesis is shown below.

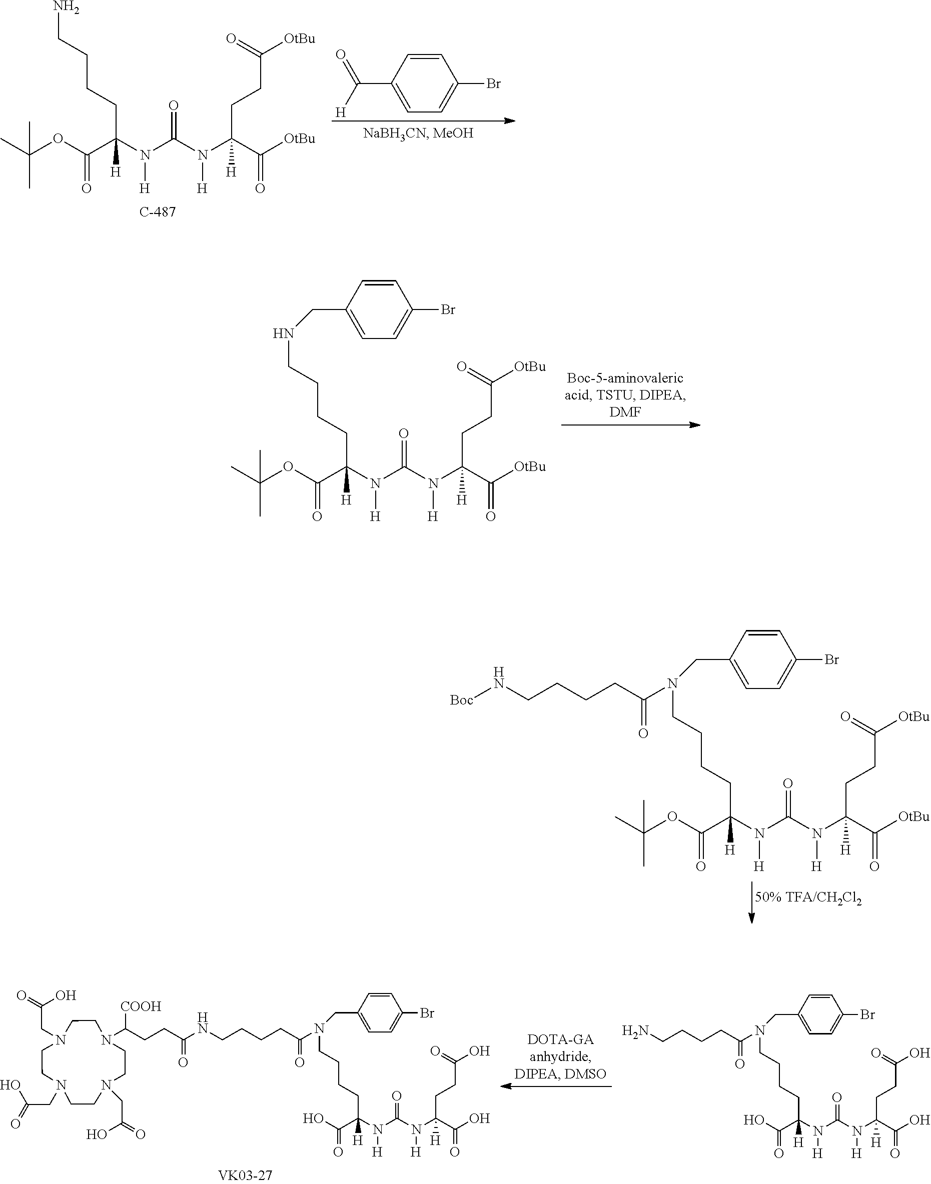 [SMILES: CC(C)(C)OC(=O)CC[C@H](NC(=O)N[C@@H](CCCCN)C(=O)OC(C)(C)C)C(=O)OC(C)(C)C ; O=Cc1ccc(Br)cc1 ; CC(C)(C)OC(=O)CC[C@H](NC(=O)N[C@@H](CCCCNCc1ccc(Br)cc1)C(=O)OC(C)(C)C)C(=O)OC(C)(C)C ; CC(C)(C)OC(=O)CC[C@H](NC(=O)N[C@@H](CCCCN(Cc1ccc(Br)cc1)C(=O)CCCCNC(=O)OC(C)(C)C)C(=O)OC(C)(C)C)C(=O)OC(C)(C)C ; O=C(O)CC[C@H](NC(=O)N[C@@H](CCCCN(Cc1ccc(Br)cc1)C(=O)CCCCNC(=O)CCC(C(=O)O)N1CCN(CC(=O)O)CCN(CC(=O)O)CCN(CC(=O)O)CC1)C(=O)O)C(=O)O ; NCCCCC(=O)N(CCCC[C@H](NC(=O)N[C@@H](CCC(=O)O)C(=O)O)C(=O)O)Cc1ccc(Br)cc1]

Table 7 shows the biodistribution of [¹⁷⁷Lu]VK-03-27 with high PSMA+PipTumor uptake and very high early renal uptake and would be a good candidate for a competitive blocker.

TABLE 7
Biodistribution (% injected
dose/gram) of [¹⁷⁷Lu]VK-03-27 in mice
bearing PSMA + PC-3 PiP and
PSMA − PC-3
flu tumors without competitive inhibitor.

| | [¹⁷⁷Lu]VK-03-27 | | |
|---|---|---|---|
| Table 7 | 2 Hour | 8 Hour | 24 Hour |
| Blood | 1.2 ± 1.7 | 0.06 ± 0.02 | 0.03 ± 0.01 |
| stomach | 0.66 ± 0.75 | 0.32 ± 0.31 | 0.14 ± 0.03 |
| spleen | 7.4 ± 3.0 | 1.2 ± 0.6 | 0.28 ± 0.13 |
| Salivary | 3.4 ± 4.4 | 0.34 ± 0.18 | 0.16 ± 0.06 |
| gland | | | |
| Lacrimal | 5.8 ± 9.6 | 0.25 ± 0.05 | 0.12 ± 0.02 |
| gland | | | |
| kidneys | 150 ± 32 | 52 ± 18 | 5.9 ± 2.7 |
| PiP | 50 ± 14 | 41 ± 9 | 49 ± 7 |
| tumor | | | |
| Flu | 5.8 ± 9.6 | 0.25 ± 0.05 | 0.12 ± 0.02 |
| tumor | | | |

Table 8 shows the biodistribution of [¹⁷⁷Lu]VK-03-27 using VK-03-27 (no Lu complex) as the CI. The CI, VK-03-27, reduces the uptake in kidneys, salivary glands and Lacrimal glands.

TABLE 8
Two-Hour Biodistribution
(% injected dose/gram)
of [¹⁷⁷Lu]VK-03-27 in mice bearing
PSMA + PC-3 PiP and PSMA − PC-3
flu tumors without and with co-injection
of 2 nmoles, 20 nmole, or 40 nmole
of competing inhibitor VK-03-27.

| | 2 Hour [¹⁷⁷Lu]VK-03-27 | | | |
|---|---|---|---|---|
| | | 2 nmole | 20 nmole | 40 nmole |
| | No CI | VK-03-27 | VK-03-27 | VK-03-27 |
| Blood | 0.43 ± 0.6 | 0.23 ± 0.5 | 0.25 ± 0.14 | 0.16 ± 0.03 |
| Stomach | 0.44 ± 0.6 | 0.23 ± 0.09 | 0.45 ± 0.35 | 0.42 ± 0.21 |
| Spleen | 8.0 ± 2.8 | 0.8 ± 0.17 | 0.39 ± 0.18 | 0.21 ± 0.1 |
| Salivary | 1.2 ± 0.25 | 0.24 ± 0.04 | 0.16 ± 0.04 | 0.25 ± 0.21 |
| Gland | | | | |
| Lacrimal | 1.06 ± 0.37 | 0.29 ± 0.08 | 0.26 ± 0.05 | 0.26 ± 0.11 |
| Glands | | | | |
| Kidneys | 140 ± 21 | 10.6 ± 0.6 | 3.5 ± 0.56 | 2.15 ± 0.1 |
| PiP | 55 ± 12 | 37 ± 8 | 26 ± 4 | 18 ± 3 |
| Tumor | | | | |
| Flu | 0.4 ± 0.02 | 0.32 ± 0.05 | 0.27 ± 0.05 | 0.25 ± 0.05 |
| Tumor | | | | |

### REFERENCES

All publications, patent applications, patents, and other references mentioned in the specification are indicative of the level of those skilled in the art to which the presently disclosed subject matter pertains. All publications, patent applications, patents, and other references (e.g., websites, databases, etc.) mentioned in the specification are herein incorporated by reference in their entirety to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.
Y. Chen et al. J. Med. Chem. 51:7933-7933, 2008);
Kiess A P, Minn I, Vaidyanathan G. Hobbs R F, Josefsson A, Shen C, Brummet M, Chen Y, Choi J, Koumarianou E, Baidoo K, Brechbiel M W, Mease R C, Sgouros G, Zalutsku M R, Pomper M G. (2S)-2-(3-(1-Carboxy-5-(4-[²¹¹At]astatobenzamido)pentyl)ureido)-pentanedioic acid for PSMA-Targeted α-Particle Radiopharmaceutical Therapy. J Nucl Med. 2016 May 26.);
International PCT patent application publication no. WO2008058192 A2, to Babich et al.;
International PCT patent application publication no. WO2016065145 A2, to Pomper et al.;
Chen et al. J. Med. Chem. 51: 7933-7943, 2008; Clinical Cancer research 17: 7645-7653, 2011);
International PCT patent application publication no. WO 2017070482 A2, to Pomper et al.;
International PCT patent application publication no. WO 2017/165473 A, to Ray et al., which is incorporated herein by reference in its entirety.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A method for imaging a prostate-specific membrane antigen (PSMA)-positive tumor or cell or treating a disease, disorder, or condition associated with PSMA, the method comprising administering to the subject a radiolabeled PSMA inhibitor in combination with a non-radiolabeled PSMA competing inhibitor, each in an amount suitable for imaging a PSMA-positive tumor or treating a disease, disorder, or condition associated with PSMA.

2. The method of claim 1, wherein the radiolabeled PSMA inhibitor is selected from the group consisting of [^{131/125}I]YC-I-27, [¹²⁵I]HS-549, [¹²⁵I]VK-02-90, [¹²⁵I]VK-02-90-Lu, [²¹¹At]VK-02-90-Lu, [¹⁷⁷Lu]VK-I-45, and [¹⁷⁷Lu]VK-03-27.

3. The method of claim 1, wherein the non-radiolabeled PSMA competing inhibitor is YC-I-27: 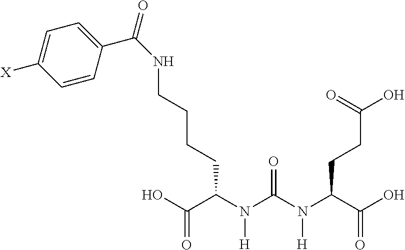
wherein X=I.

4. The method of claim 1, wherein the non-radiolabeled PSMA competing inhibitor is an analog of YC-I-27: 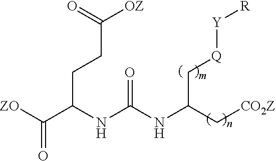
wherein:
R is a C₆-C₁₂ substituted or unsubstituted aryl, a C₆-C₁₂ substituted or unsubstituted heteroaryl, a C₁-C₆ substituted or unsubstituted alkyl, or —NR′R′:
Q is C(O), O, NR′, S, S(O)₂, C(O)₂, or (CH₂)ₚ;
Y is C(O), O, NR′, S, S(O)₂, C(O)₂, or (CH₂)ₚ;
Z is H or C₁-C₄ alkyl;
m is 0, 1, 2, 3, 4 or 5;
n is 0, 1, 2, 3, 4, 5 or 6;
p is 0, 1, 2, 3, 4, 5 or 6;
R′ is H, C(O), S(O)₂, C(O)₂, a C₆-C₁₂ substituted or unsubstituted aryl, a C₆-C₁₂ substituted or unsubstituted heteroaryl or a C₁-C₆ substituted or unsubstituted alkyl, when substituted, aryl, heteroaryl and alkyl are substituted with halogen, C₆-C₁₂ heteroaryl, —NR′R′ or COOZ;
further wherein: (i) at least one of R or R′ is a C₆-C₁₂ aryl or C₆-C₁₂ heteroaryl substituted with a halogen or (ii) at least one of R or R′ is a C₆-C₁₂ heteroaryl;
or a pharmaceutically acceptable salt thereof.

5. The method of claim 4, wherein:
n is 0 or 1;
m is 0, 1, 2, 3 or 4;
Q is NR′;
Y is C(O) or CH₂; and
R is a C₆-C₁₂ substituted or substituted aryl.

6. The method of claim 5, wherein R is a phenyl moiety substituted with a halogen.

7. The method of claim 1, wherein the non-radiolabeled PSMA competing inhibitor is a compound of formula (XX-A): 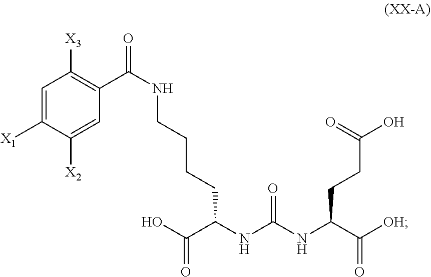
wherein:
X₁ and X₂ are each independently selected from the group selected from Br, Cl, I, F, H, —OCH₃, —OCH₂CH₃, —OCH(CH₃)₂, and —OC(CH₃)₃.

8. The method of claim 7, wherein the compound of formula (XX-A) is selected from the group consisting of compounds 1-23:
Compounds 1-23

| Compound | X₁ | X₂ | X₃ |
|---|---|---|---|
| 1 | Br | H | H |
| 2 | Cl | H | H |
| 3 | F | H | H |
| 4 | H | H | H |
| 5 | —OCH₃ | H | H |
| 6 | —OCH₂CH₃ | H | H |
| 7 | —OCH(CH₃)₂ | H | H |
| 8 | —OC(CH₃)₃ | H | H |
| 9 | H | I | H |
| 10 | H | Br | H |
| 11 | H | Cl | H |
| 12 | H | F | H |
| 13 | H | —OCH₃ | H |
| 14 | H | —OCH₂CH₃ | H |
| 15 | H | —OCH(CH₃)₂ | H |
| 16 | H | —OC(CH₃)₃ | H |
| 17 | Br | H | F |
| 18 | I | H | F |
| 19 | Cl | H | F |
| 20 | —OCH₃ | H | F |
| 21 | —OCH₂CH₃ | H | F |
| 22 | —OCH(CH₃)₂ | H | F |
| 23 | —OC(CH₃)₃ | H | F |

9. The method of claim 1, wherein the non-radiolabeled PSMA competing inhibitor is a reverse carbamate of formula (XX-B): 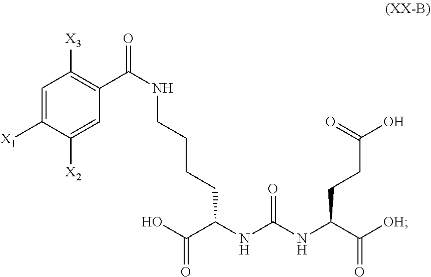
wherein:
X₃ is F;
X₂ is H; and
X₁ is selected from the group consisting of Br, Cl, I, —OCH₃, —OCH₂CH₃, —OCH(CH₃), and —OC(CH₃)₃.

10. The method of claim 9, wherein the compound of formula (XX-B) is selected from the group consisting of compounds 24-30:
Compounds 24-30

| Compound | X₁ | X₂ | X₃ |
|---|---|---|---|
| 24 | I | H | F |
| 25 | Br | H | F |
| 26 | Cl | H | F |
| 27 | —OCH₃ | H | F |
| 28 | —OCH₂CH₃ | H | F |
| 29 | —OCH(CH₃)₂ | H | F |
| 30 | —OC(CH₃)₃ | H | F |

11. The method of claim 1, wherein the non-radiolabeled PSMA competing inhibitor is a halo-nicotinamide of formula (XX-C): 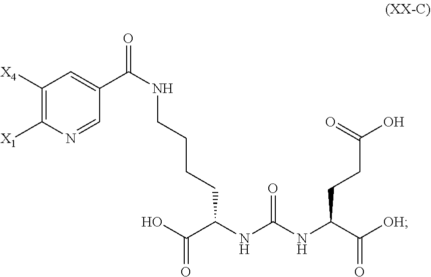
Wherein:
X₁ and X₄ are each independently selected from the group consisting of H, I, Br, Cl, and F.

12. The method of claim 11, wherein the compound of formula (XX-C) is selected from the group consisting of:

| Compound | X₁ | X₄ |
|---|---|---|
| 31 | H | I |
| 32 | H | Br |
| 33 | H | Cl |
| 34 | H | F |
| 35 | I | H |
| 36 | Br | H |
| 37 | Cl | H |
| 38 | F | H |

13. The method of claim 1, wherein the non-radiolabeled PSMA competing inhibitor is selected from the group consisting of:

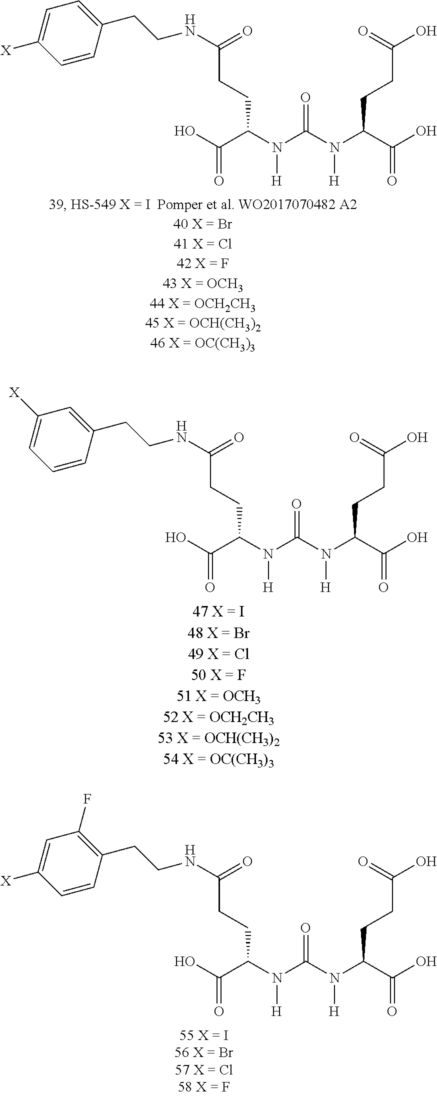

14. The method of claim 1, wherein the non-radiolabeled PSMA competing inhibitor is selected from the group consisting of: PSMA-620, PSMA-904, and analogs thereof.

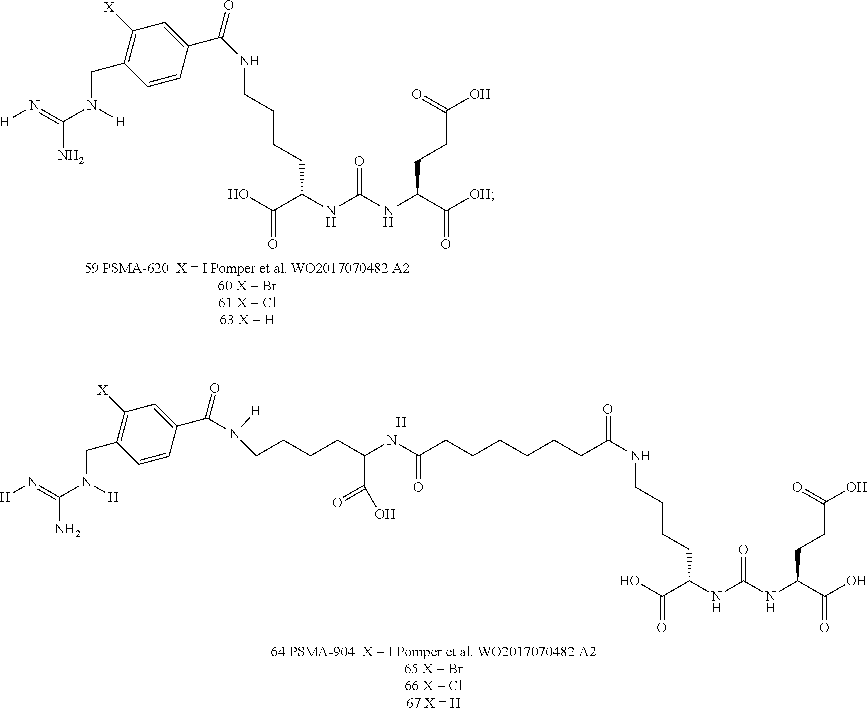

15. The method of claim 1, wherein the non-radiolabeled PSMA competing inhibitor is selected from the group consisting of: VK-02-90, VK-01-45, and VK-01-45-Lu, and VK-03-27.

16. The method of claim 1, wherein the prostate-specific membrane antigen (PSMA)-positive tumor or cell is selected from the group consisting of: a prostate tumor or cell, a metastasized prostate tumor or cell, a lung tumor or cell, a renal tumor or cell, a glioblastoma, a pancreatic tumor or cell, a bladder tumor or cell, a sarcoma, a melanoma, a breast tumor or cell, a colon tumor or cell, a germ cell, a pheochromocytoma, an esophageal tumor or cell, a stomach tumor or cell, and combinations thereof.

17. The method of claim 1, wherein the prostate-specific membrane antigen (PSMA)-positive tumor or cell is primary clear cell renal carcinoma.

18. The method of claim 1, wherein the method further comprises taking an image.

19. The method of claim 18, wherein the taking of an image is selected from the group consisting of positron emission tomography (PET) and single-photon emission computed tomography (SPECT).

20. A pharmaceutical composition comprising a radiolabeled PSMA inhibitor in combination with a non-radiolabeled PSMA competing inhibitor.

21. A kit comprising a radiolabeled PSMA inhibitor in combination with a non-radiolabeled PSMA competing inhibitor.
